(19) United States
(12) Patent Application Publication
(10) Pub. No.: US 2014/0335108 A1
(43) Pub. Date: Nov. 13, 2014

(54) UTILITY OF INSULIN-LIKE 6 (INSL6) FOR THE TREATMENT OF AUTOIMMUNE DISEASES

(71) Applicant: TRUSTEES OF BOSTON UNIVERSITY, Boston, MA (US)

(72) Inventors: Kenneth Walsh, Carlisle, MA (US); Ling Zeng, Brighton, MA (US)

(21) Appl. No.: 14/364,343

(22) PCT Filed: Dec. 12, 2012
(86) PCT No.: PCT/US2012/069071

§ 371 Date: Jun. 11, 2014

Related U.S. Application Data
(60) Provisional application No. 61/569,499, filed on Dec. 12, 2011.

(51) Int. Cl. A61K 47/48; A61K 38/17; C07K 14/47

(52) U.S. Cl. CPC A61K 47/48415; C07K 14/47; A61K 38/1709
USPC 424/178.1; 514/21.2; 514/17.9; 514/16.6; 514/7.3; 530/402; 530/391.7; 536/23.5; 435/320.1; 435/354

(57) ABSTRACT

The present invention is directed to compositions and methods to treat an autoimmune disease in a subject, comprising an insulin-like 6 (Insl6) agent, such as an Insl6 polypeptide or variant or fragment thereof, or a nucleic acid encoding Insl6 poly peptide or variant or fragment thereof. Aspects of the present invention relate to use of Insl6 agents to reduce T-regulatory (Treg) cells in the subject and to reduce pro-inflammatory cytokines in a subject with an autoimmune disease such as a muscle autoimmune disease. The present invention also relates to methods and kits for the treatment of autoimmune diseases in a subject, and methods to diagnose a subject with an autoimmune disease such as myositis.

### CROSS REFERENCE TO RELATED APPLICATION

This application claims benefit under 35 U.S.C. §119(e) of the U.S. provisional application No. 61/569,499 filed Dec. 12, 2011, the contents of which is incorporated herein by reference in their entirety.

### GOVERNMENT SUPPORT

This invention was made with U.S. Government Support under Contract Number AG034972 awarded by the National Institutes of Health (NIH). The Government has certain rights in the invention.

### SEQUENCE LISTING

The instant application contains a Sequence Listing which has been submitted in ASCII format via EFS-Web and is hereby incorporated by reference in its entirety. Said ASCII copy, created on Dec. 11, 2012, is named 72631PCT.txt and is 5,596 bytes in size.

### FIELD OF THE INVENTION

The present invention is directed to compositions and methods to treat an autoimmune disease in a subject, comprising an insulin-like 6 (Insl6) agent, such as an Insl6 polypeptide or variant or fragment thereof, or a nucleic acid encoding Insl6 polypeptide or variant or fragment thereof. The present invention also relates to methods and kits for the treatment of autoimmune diseases in a subject, and methods to diagnose a subject with myositis.

### BACKGROUND OF THE INVENTION

Myositis describes a cluster of inflammatory muscle diseases of unknown origin. Myositis is a T cell-mediated autoimmune disease that is associated with alterations in regulatory T cells (Y. Allenbach 2009 Am J Pathol 174:989). Both polymyositis (PM) and dermatomyositis (DM) cause muscle weakness; where DM is also associated with a skin rash. In addition, the related disease inclusion body myositis (IBM) develops later in life and has a worse prognosis. These diseases are chronic and persistent. There are no know cures for these PM and DM, but the symptoms are typically treated with corticosteroids. Most individuals with DM and PM respond to therapy, but those severe disease are refractory to treatment and suffer significant disability. There is no known effective therapy for IBM.

### SUMMARY OF THE INVENTION

The present invention generally relates to compositions and methods for the treatment of a subject determined to have an autoimmune disease or an immune related disease or disorder, where the subject is administered a composition comprising an insulin-like 6 (Insl6) protein or functional fragment thereof, or any variant or homologue of Insl6. In some embodiments, the Insl6 polypeptide is conjugated to a fusion partner, e.g., a fusion protein which increase the stability of the Insl6 protein. In some embodiments, the fusion partner is an IgG1 Fc, e.g., human IgG1 Fc.

The present invention is based upon the discovery that Insl6, or a fusion protein or a functional fragment, homologue or variant thereof can protect muscle dysfunction in vivo in a mouse model of C protein-induced (poly)mytosis (CIM), and reduces muscle inflammation in vivo. The inventors have also demonstrated that administration of Insl6 protein, in particular Insl6 protein fused to Fc protects against muscle dysfunction in a mouse model of an autoimmune disease, e.g., in the CIM myositis model. Furthermore, the inventors have demonstrated that in human subjects suffering from myositis, the level of Insl6 transcript and/or protein expression is decreased as compared to normal subjects.

Furthermore, the inventors have also discovered that the Insl6 protein or functional fragment thereof, or any variant or homologue of Insl6, can regulate T-regulatory (T_{reg}) cells in tissues, in particular, conditioned media comprising Insl6 can modulate, e.g., decrease or reduce T-regulatory cells in vitro. Accordingly, the compositions and methods as disclosed herein can be used to treat a subject determined to have an autoimmune disease or immune-related disease or disorder, in particular, in some embodiments, the autoimmune disease is a disease with elevated T-regulatory cells, wherein the Insl6 protein or fragment thereof can be used to reduce T_{reg} cell and/or B cell activation in such autoimmune disease and/or immune-related diseases and disorders.

Accordingly, the present invention relates to a method of using an Insl6 gene product, (e.g., the nucleic acid encoding Insl6 or a polypeptide) or a functional fragment or derivative or variant thereof to treat an autoimmune disease. In some embodiments, the present invention relates to a method to treat autoimmune diseases, in particular autoimmune diseases of the muscle, such as, for example, muscle inflammation and/or myositis. In some embodiments, the autoimmune disease is selected from any or a combination of autoimmune myocarditis, lupus, diabetes, multiple sclerosis and the like, or any autoimmune disease with elevated levels of T-regulatory cells and/or abnormal amounts of autoantibodies.

In further embodiments, the level of Insl6 expression in a subject can be used to identify a subject with having, or an increased risk of developing a muscle inflammatory disease, such as, but not limited to myositis.

One aspect of the present invention relates to a method for treating an autoimmune disease in a subject comprising administering to the subject a composition comprising a insulin-like 6 (Insl6) protein or fragment thereof.

Another aspect of the present invention relates to a method for treating an autoimmune disease in a subject comprising administering to the subject determined to have an autoimmune disease a composition comprising a insulin-like 6 (Insl6) protein or fragment thereof, and wherein the composition is not administered to a subject where the subject is not determined to have an autoimmune disease.

In some embodiments of all aspects of the present invention, the autoimmune disease is myositis, such as, but not limited to, polymyositis (PM), dermatomyositis (DM) or inclusion body myositis (IBM). In some embodiments, the autoimmune disease is a T-cell mediated autoimmune disease.

In some embodiments of all aspects of the present invention, a fragment of insulin-like 6 protein is a biologically active fragment of Insl6. In some embodiments of all aspects of the present invention, an insulin-like 6 (Insl6) protein is fused to a carrier protein, such as, but not limited to, where an insulin-like 6 (Insl6) protein is fused to a Fc. In some embodiments of all aspects of the present invention, an insulin-like 6 (insl6) protein is encoded by nucleic acid present in a vector, for example, but not limited to a viral vector and the like.

In some embodiments of all aspects of the present invention, a composition used in the methods as disclosed herein further comprises an additional therapeutic agent to treat myositis, e.g., but not limited to, a corticosteroid or similar agent.

In some embodiments of all aspects of the present invention, an insulin-like 6 (Insl6) protein or fragment thereof reduces at least one inflammatory protein selected from the group consisting of: CD4, CD8, CD11b, TNFα and MCP-1. In some embodiments, an insulin-like 6 (Insl6) protein or fragment thereof of the present invention can be used in a method to treat an inflammatory disease or disorder, e.g., but not limited to sepsis.

In some embodiments of all aspects of the present invention, an insulin-like 6 (Insl6) protein or fragment thereof can be administrated by any one or a combination of topical administration, parenteral administration, enteral oral, parenteral (e.g., intravenous, subcutaneous or intramuscular), rectal, intracisternal, intravaginal, intraperitoneal, ocular, or nasal routes.

Another aspect of the present invention relates to a fusion protein comprising: (a) an insulin-like 6 (Insl6) polypeptide or fragment thereof, wherein said fragment has at least 95% amino acid sequence identity to a portion of the Insl6 protein and is at least 6 amino acids; and (b) a first fusion partner, wherein said first fusion partner is conjugated to said Insl6 polypeptide or fragment thereof. In some embodiments of all aspects of the present invention, a first fusion partner is fused to the N-terminus or to the C-terminus of the Insl6 protein or Insl6 fragment, and can be, for example, a IgG1 Fc, e.g., human IgG1 Fc.

In some embodiments of all aspects of the present invention, an Insl6 fragment is a soluble fragment. In some embodiments, an Insl6-Fc fusion protein can optionally further comprise a second fusion partner. In some embodiments of all aspects of the present invention, conjugation of Insl6 protein or fragment thereof to a first fusion protein is a covalent bond.

In some embodiments of all aspects of the present invention, an insl6 protein or Insl6 fragment lacks the N-terminal signal sequence. The N-terminal signal sequence of Insl6 is amino acids 1-20 of SEQ ID NO:1. In some embodiments of all aspects of the present invention, an Insl6 protein is a human Insl6 protein, and corresponds to amino acid SEQ ID NO: 1, or a functional variant or functional derivative or functional fragment of SEQ ID NO: 1. In some embodiments, a functional fragment of an Insl6 protein comprises at least the A-domain of Insl6 (amino acids 173-198 of SEQ ID NO:1) and/or at least the B-domain of Insl6 (amino acids 55-168 of SEQ ID NO:1). In some embodiments, an Insl6 protein or an insl6 functional fragment is a fusion protein which has an amino acid sequence with at least 95% identity to the sequence of SEQ ID NO: 1, or a functional derivative or functional variant thereof, or alternatively, can have an amino acid sequence comprising, or consisting essentially of, the sequence of SEQ ID NO: 1, or a functional derivative or functional variant thereof. In some embodiments of all aspects of the present invention, an Insl6 protein or fragment thereof, or a fusion protein thereof has enhanced proteolytic stability.

In some embodiments of all aspects of the present invention, an Insl6 protein or fragment thereof, or a fusion protein thereof can be used in a method to treat an in inflammatory disorder or an autoimmune disease, e.g., but not limited to myositis, e.g., polymyositis (PM), dermatomyositis (DM) or inclusion body myositis (IBM).

Another aspect of the present invention relates to a pharmaceutical composition comprising an Insl6 protein or fragment thereof, or a fusion protein thereof and a pharmaceutically acceptable carrier.

Another aspect of the present invention relates to a method for producing an Insl6-fusion protein comprising: (a) introducing into a cell with a vector comprising a sequence encoding the fusion protein operably linked to a promoter, wherein the fusion protein is insulin-like 6 (Insl6) polypeptide or fragment thereof linked to a first fusion protein; and (b) culturing said cell under conditions where said protein is expressed. In some embodiments, the method further comprises purifying the Insl6-fusion protein of step (b).

Another aspect of the present invention relates to a polynucleotide encoding the fusion protein, wherein the polynucleotide encodes an insulin-like 6 (Insl6) polypeptide or fragment thereof which has at least 85% amino acid sequence identity to a portion of the insulin-like 6 (Insl6) polypeptide or fragment thereof protein; and a first fusion partner.

Another aspect of the present invention relates to a vector comprising the polynucleotide encoding an insl6 fusion protein, wherein the Insl6 polypeptide can be a fragment thereof, and can be linked to a first fusion protein, e.g., a Fc or fragment thereof. In some embodiments, the vector is a viral vector, e.g., but not limited, to any one or a combination selected from an adenoviral vector, a poxvirus vector and a lentiviral vector.

In some embodiments, a the nucleic acid sequence which encodes an insulin-like 6 (Insl6) polypeptide or fragment thereof which has at least 95% amino acid sequence identity to a portion of the Insl6 protein; and a first fusion partner, wherein the nucleic acid sequence is operatively linked to tissue- or cell-type specific promoter, e.g., a muscle specific promoter.

In some embodiments of all aspects of the present invention, a pharmaceutical composition comprising the vector further comprises a pharmaceutically acceptable carrier. Another aspect of the present invention relates to a host cell comprising the vector as disclosed herein.

Another aspect of the present invention relates to a method for treating a subject having a muscle inflammatory disorder or an autoimmune disease, said method comprising administering to said subject an Insl6-fusion protein as disclosed herein in an amount effective to treat said patient, e.g., to reduce at least one symptom of the autoimmune disease. In some embodiments, the muscle inflammatory disorder is myositis, e.g., but not limited to, polymyositis (PM), dermatomyositis (DM) or inclusion body myositis (IBM).

### BRIEF DESCRIPTION OF THE DRAWINGS

FIGS. 1A-1D show C protein-induced (poly)myositis model (CIM). FIG. 1A is a scheme of recombinant human myosin binding protein C fragment 2 purification. The fragment was amplified by PCR from human skeletal muscle cDNA; and sub-cloned into the pQE-30 vector that fuses the C protein fragment to a poly-histidine sequence. The His-tagged protein was expressed in E. coli and purified with nickel resin. The purified protein was analyzed on SDS PAGE and stained with coomassie blue, and appeared as a 37 kDa band. FIG. 1A discloses “6×His” as SEQ ID NO: 4. FIG. 1B shows a scheme of the CIM model. C57BL/6 female mice were immunized intradermally with recombinant human myosin binding protein C fragment 2 (1.5 mg) or an equal volume of PBS (control) emulsified with Freund's adjuvant. 2 μg of pertussis toxin (PT) in PBS was injected intraperitoneally at the same time. Muscle function and tissue histology was typically investigated 14 days after immunization. FIG. 1C shows a representative image of H&E-stained tibialis anterior (TA) muscle sections between 14 and 21 days after the immunization. Mononuclear cell infiltration was found in the perimysial (surrounding myofibers) and perivascular (surrounding vessels) sites, but rarely seen in endomysial sites (within myofibers). FIG. 1D shows gastrocnemius muscle lysate from control and experimental autoimmune mysositis mice were analyzed by western immunoblotting for Insl6 protein expression. Tubulin was used as a loading control. A representative image is shown. Insl6 protein expression upregulated expression in this model at 14 days.

FIGS. 2A-2D show Insl6 transgenic mice are protected against muscle dysfunction in the CIM myositis model. Immunogen was injected into muscle-specific Insl6 transgenic (TG) female mice and their wild-type (WT) littermates as described in FIG. 1. FIG. 2A shows a wild type mouse (WT#1) and FIG. 2B shows a Insl6 transgenic mouse (TG) on the rotarod treadmill apparatus. FIG. 2C shows muscle function reported as running distance, FIG. 2D shows muscle function reported as rotarod performance before and 14 days after the immunization.

FIGS. 3A-3E show Insl6 overexpression attenuates muscle inflammation in the CIM model. Wild-type (WT) and Insl6 transgenic (TG) mice were immunized as described in the CIM protocol (see FIG. 1). At 14 days post-immunization, mice were sacrificed and total RNA was isolated from TA muscles and subject to cDNA synthesis. Relative transcript expression of the T cell markers CD4 (FIG. 3D) and CD8 (FIG. 3E), the macrophage marker CD11b (FIG. 3C), and the pro-inflammatory cytokines TNF-α (FIG. 3A) and MCP-1 (FIG. 3B) were measured by qRT-PCR (n=7-9 in each experimental group). The results are presented as the mean±SEM.

FIGS. 4A-4C show the construction of Fc-Insl6 fusion plasmid vector and outline of hydrodynamic plasmid delivery. FIG. 4A shows the full length murine Insl6 cDNA was sub-cloned into pLEV113 vector (LakePharma), such that the human Fc fragment was located upstream of the Insl6 peptide sequence. FIG. 4B shows the method of hydrodynamic tail vein delivery of the Fc-Insl6 plasmid or control plasmid according to standard protocol. FIG. 4C shows the serum Fc concentration was detected by ELISA according manufacture's protocol (LakePharma).

FIGS. 5A-5B shows results of hydrodynamic delivery of a plasmid expressing the Fc-Insl6 fusion protein protects mice from muscle dysfunction in the CIM myositis model. Mice were immunized as described in FIG. 1. The day after immunization, control and Fc-Ins16-expressing plasmids were delivered by hydrodynamic tail vein injection. FIG. 5A shows muscle function was evaluated by rotarod and FIG. 5B shows muscle function as evaluated by treadmill immediately prior to the immunization or at 14 days after the immunization/plasmid delivery. For treadmill testing, the slope is 0 degrees (horizontal) and mice are acclimatized by running at 6 meters per minute for 5 minutes. To determine running distance, the treadmill speed was increased from the acclimatization speed by 1 meters per minute every five minutes, and the running distance until exhaustion is recorded. For the Rotarod test, mice are acclimatized at 10 rpm for 5 minutes. Following acclimatization, the length of time on the Rotarod is recorded when it is run at 15 rpm (a score of 1 is defined as 30 seconds on the Rotarod for 30 seconds at 15 rpm). The results are presented as the mean±SEM. N=3*p<0.05.

FIGS. 6A-6B show Insl6 transcript levels are reduced in myositis patients. FIG. 6A shows Insl6 transcript expression in a Myositis patient (PM, polymyositis; DM, dermatomyositis) muscle cDNA as compared to control muscle sample. Relative transcript expression of Insl6 was measured by qRT-PCR. FIG. 6B shows the relative Insl6 transcript expression for age and gender matched subjects.

### DETAILED DESCRIPTION OF THE INVENTION

The present invention generally relates to the treatment of a subject determined to have an autoimmune disease or an immune-related disease or disorder, a composition comprising an insulin-like 6 (Insl6) protein or functional fragment thereof, or any variant or homologue of Insl6. In some embodiments, the Insl6 polypeptide is conjugated to a fusion partner, e.g., a fusion protein which increase the stability of the Insl6 protein. In some embodiments, the fusion partner is an IgG1 Fc, e.g., human IgG1 Fc.

The present invention is based upon the discovery that Insl6, or a fusion protein or a functional fragment, homologue or variant thereof can protect muscle dysfunction in vivo in a mouse model of C protein-induced (poly)mytosis (CIM), and reduces muscle inflammation in vivo. The inventors have also demonstrated that administration of Insl6 protein, in particular Insl6 protein fused to Fc protects against muscle dysfunction in a mouse model of an autoimmune disease, e.g., in the CIM myositis model. Furthermore, the inventors have demonstrated that in human subjects suffering from myositis, the level of Insl6 transcript and/or protein expression is decreased as compared to normal subjects.

Furthermore, the inventors have also discovered that the Insl6 protein or functional fragment thereof, or any variant or homologue of Insl6, can regulate T-regulatory (T_{reg}) cells in tissues, in particular, conditioned media comprising Insl6 can modulate T-regulatory cells in vitro. Accordingly, the compositions and methods as disclosed herein can be used to treat a subject determined to have an autoimmune disease or immune-related disease or disorder, in particular, in some embodiments, the autoimmune disease is a disease with abnormal levels of T-regulatory cells, wherein the Insl6 protein or fragment thereof can be used to reduce T_{reg} cell and/or B cell activation in such autoimmune disease and/or immune-related diseases and disorders.

Accordingly, the present invention relates to a method of using an Insl6 gene product, (e.g., the nucleic acid encoding Insl6 or a polypeptide) or a functional fragment or derivative or variant thereof to treat an autoimmune disease. In some embodiments, the present invention relates to a method to treat autoimmune diseases, in particular autoimmune diseases of the muscle, such as, for example, muscle inflammation and/or myositis. In some embodiments, the autoimmune disease is selected from any or a combination of autoimmune myocarditis, lupus, diabetes, multiple sclerosis and the like, or any autoimmune disease with abnormal levels of T-regulatory cells and/or elevated levels of autoantibodies.

In further embodiments, the level of Insl6 expression in a subject can be used to identify a subject with having, or an increased risk of developing a muscle inflammatory disease, such as, but not limited to myositis.

Accordingly, in one embodiment, the present invention provides methods and compositions to decrease muscle inflammation in a subject. For example, the methods of the present invention also provide methods and compositions to treat a subject at risk of developing, or having a muscle autoimmune disease, e.g., myositis or a subject in need of treatment, where the subject has been determined to have myositis or other muscle inflammatory or autoimmune disease. In such an embodiment, the method comprises administration of an effective amount of a pharmaceutical composition comprising an agent that functions as an agonist, for example but not limited to agents that activate and/or increases the gene expression of the Insl6 protein or functional fragment thereof, or any variant or homologue of Insl6 to a subject in need thereof. In some embodiments, the pharmaceutical compositions comprise Insl6 protein or functional fragment thereof, or any variant or homologue of Insl6. In some embodiments, the Insl6 protein can be fused to a fusion partner, e.g., but not limited to, a Fc protein or fragment thereof. In an alternative embodiment, the present invention provides a means to treat a subject with, or at risk of developing myositis or other muscle inflammatory or autoimmune disease by administering a pharmaceutical composition comprising a nucleic acid encoding Insl6 (SEQ ID: NO: 1) or a homologue or variant, or fragment thereof to the subject.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

By “insulin-like 6” or “Insl6” is meant a polypeptide having an amino acid sequence at least about 60%, or at least about 70%, or at least about 80%, or at least about 90%, or at least about 95%, or at least about 96%, or at least about 97%, or at least about 98%, or at least about 99% identical to SEQ ID NO: 1.

The term “wild type” refers to the naturally-occurring polynucleotide sequence encoding a protein, or a portion thereof, or protein sequence, or portion thereof, respectively, as it normally exists in vivo. Accordingly, as disclosed herein, the wild type amino acid sequence for the proprotein of human Insl6 protein corresponds to SEQ ID NO: 1. The protein of SEQ ID NO: 1 can be post-translationally processed in vivo to comprise the B-domain (amino acids 21-53 of SEQ ID NO:1) and the A-domain (amino acids 173-198 of SEQ ID NO:1), and optionally the C-domain (amino acids 55-168 of SEQ ID NO:1).

The term “soluble Insl6 polypeptide” as used herein refers to a Insl6 polypeptide that does not comprise at least part of, or all of, the amino acids which allow it to functionally bind to the membrane.

By a “polynucleotide encoding Insl6” is meant a polynucleotide encoding a polypeptide having at least about 60%, or at least about 70%, or at least about 80%, or at least about 90%, or at least about 95%, or at least about 96%, or at least about 97%, or at least about 98%, or at least about 99% sequence identity to any of the amino acid sequences corresponding to SEQ ID NO: 1.

By “human Fc” is meant a polypeptide with an amino acid sequence at least about 50%, or at least about 60%, or at least about 70%, or at least about 80%, or at least about 90%, or at least about 95%, or at least about 96%, or at least about 97%, or at least about 98%, or at least about 99% identical to the sequence of SEQ ID NO: 3.

By a “polynucleotide encoding human Fc” is meant a polynucleotide that encodes a polypeptide sequence having at least about 50%, or at least about 60%, or at least about 70%, or at least about 80%, or at least about 90%, or at least about 95%, or at least about 96%, or at least about 97%, or at least about 98%, or at least about 99% identity to the sequence of SEQ ID NO: 3.

The term “mutant” refers to any change in the genetic material of an organism, in particular a change (i.e., deletion, substitution, addition, or alteration) in a wild-type polynucleotide sequence or any change in a wild-type protein sequence. The term “variant” is used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent). The term mutation is used interchangeably herein with polymorphism in this application.

As used herein, “muscle” or “muscle cell” refers to any cell that contributes to muscle tissue. Myoblasts, satellite cells, myotubes, and myofibril tissues are all included in the term “muscle cells”. Muscle cells may include those within skeletal, cardiac and smooth muscles.

As used herein, the terms “autoimmune disease” or “auto-immune disease” and “autoimmune-related disease” are used interchangeably, and refer to a disease or condition that occurs when the body tissues are attacked by its own immune system. The immune system is a complex organization within the body that is designed normally to “seek and destroy” invaders of the body, including infectious agents. Subjects with autoimmune diseases frequently have unusual antibodies circulating in their blood that target their own body tissues. Autoimmune diseases also include diseases with immunoregulatory abnormalities, such as for example a wide variety of autoimmune and chronic inflammatory diseases, including systemic lupus erythematosis, chronic rheumatoid arthritis, type 1 diabetes mellitus, inflammatory bowel disease, biliary cirrhosis, uveitis, multiple sclerosis, graft versus host (GVH) disease and other disorders such as Crohn's disease, ulcerative colitis, bullous pemphigoid, sarcoidosis, psoriasis, ichthyosis, and Graves ophthalmopathy. Although the underlying pathogenesis of each of these conditions can be quite different, they have in common the appearance of a variety of autoantibodies and self-reactive lymphocytes, and are therefore auto-immune disease which can be treated with the compositions and methods as disclosed herein. Autoimmune diseases are also referred to in the art as intractable diseases and allergic diseases. As used herein, “allergic disease” refers to a disease associated with allergic reaction. Specific examples include chronic bronchitis, atopic dermatitis, pollinosis (allergic rhinitis), allergic angiitis, allergic conjunctivitis, allergic gastroenteritis, allergic hepatopathy, allergic cystitis, and allergic purpura. Without being bound by theory, as used herein the term “immune disease” refers to a disease resulting from dysfunction of the immune system, one of defense mechanisms in the body, including diseases caused by both abnormal humoral and cellular immunity. This term also includes autoimmune diseases caused by autoantibody, autosensitized lymphocyte or immune complex, as well as graft versus host disease caused by graft versus host reaction (GVH reaction) in which graft rejection occurs, as well as allergic diseases. As used herein, auto-immune disease also include “Th1-dominant autoimmune diseases” which are autoimmune disease showing increased cytokine production from Th1 cells, including IFN-γ, IL-2, GM-CSF, TNF-α, and IL-3. Specific examples of such Th1-dominant autoimmune diseases include, for example but are not limited to, multiple sclerosis, insulin-dependent diabetes mellitus, Crohn's disease, uveitis, chronic rheumatism, and systemic lupus erythematosus. As used herein, the term autoimmune diseases also encompass “autoimmune diseases not known to be Th 1-dominant” which are autoimmune disease that is not known to show increased cytokine production from Th1 cells. Specific examples include scleroderma, multiple myositis, vasculitis syndrome, mixed connective tissue disease, Sjogren's syndrome, hyperthyroidism, Hashimoto's disease, myasthenia gravis, Guillain-Barre syndrome, autoimmune hepatopathy, ulcerative colitis, autoimmune nephropathy, autoimmune hematopathy, idiopathic interstitial pneumonia, hypersensitivity pneumonitis, autoimmune dermatosis, autoimmune cardiopathy, autoimmune infertility, and Behcet's disease. One commonly known auto-immune disease is graft versus host disease caused by graft versus host reaction (GVH reaction) in which graft rejection occurs. In subjects that are recipients of organ transplantation, such as bone marrow transplantation, the host lymphocytes recognize the foreign tissue antigens and begin to produce antibodies which lead to graft rejection. Accordingly, subjects that are recipients of bone marrow transplants or organ transplants, either before transplantation, at the time of transplantation or post transplantation can be treated with the compositions and methods as disclosed herein. Accordingly, the methods and compositions as disclosed herein Insl6 agents as disclosed herein, e.g., Insl6 proteins or nucleic acid encoding Insl6 proteins can be administered to subjects in need of, or scheduled to receive immune suppression (i.e. in need of immunosuppressive therapy), for example subjects who are organ or bone marrow transplant recipients or subjects having, or at risk of developing an auto-immune disease.

As used herein, the term “immunosuppressive agents” is meant any composition capable of suppressing the immune system, and includes analogs, hydrolysis products, metabolites, and precursors of an immunosuppressive agent unless the context precludes it. In some embodiments, immunosuppressive agents useful in the compositions and methods as disclosed herein can be selected from one of the following compounds: mycophenolic acid, cyclosporin, azathioprine, tacrolimus, cyclosporin A, FK506, rapamycin, leflunomide, deoxyspergualin, prednisone, azathioprine, mycophenolate mofetil, OKT3, ATAG or mizoribine. One example of such a composition is cyclosporine.

The term “inflammatory disorders” or “inflammatory diseases” as used herein refer to disorders or conditions mediated by an inflammatory cytokine cascade, defined herein as an in vivo release from cells of at least one pro-inflammatory cytokine in a subject, wherein the cytokine release affects a physiological condition of the subject. Non limiting examples of cells that produce proinflammatory cytokines are monocytes, macrophages, neutrophils, epithelial cells, osteoblasts, fibroblasts, smooth muscle cells, and neurons.

As used herein, a “cytokine” is a generic term for proteins released by any of the lymph cells that act on other cells as intercellular mediators and affect cellular activity and control inflammation. Cytokines are typically soluble proteins or peptides which are naturally produced by mammalian cells and which act in vivo as humoral regulators at micro- to picomolar concentrations. Cytokines can, either under normal or pathological conditions, modulate the functional activities of individual cells and tissues. A proinflammatory cytokine is a cytokine that is capable of causing any of the following physiological reactions associated with inflammation: vasodialation, hyperemia, increased permeability of vessels with associated edema, accumulation of granulocytes and mononuclear phagocytes, or deposition of fibrin. In some cases, the pro-inflammatory cytokine can also cause apoptosis, such as in chronic heart failure, where TNF has been shown to stimulate cardiomyocyte apoptosis. Nonlimiting examples of pro-inflammatory cytokines are tumor necrosis factor (TNF), interleukin (IL)-1.alpha., IL-1.beta., IL-6, IL-8, IL-18, interferon-γ (INFγ), HMG-1, platelet-activating factor (PAF), and macrophage migration inhibitory factor (MIF). In preferred embodiments of the invention, the pro-inflammatory cytokine that is inhibited by cholinergic agonist treatment is TNF, an IL-1, IL-6 or IL-18, because these cytokines are produced by macrophages and mediate deleterious conditions for many important disorders, for example endotoxic shock, asthma, rheumatoid arthritis, inflammatory bile disease, heart failure, and allograft rejection. In most preferred embodiments, the pro-inflammatory cytokine is TNF. Pro-inflammatory cytokines are to be distinguished from anti-inflammatory cytokines, such as IL-4, IL-10, and IL-13, which are not mediators of inflammation. In some embodiments, release of anti-inflammatory cytokines is not inhibited by cholinergic agonists. Additionally examples of cytokines include, lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance (MIS); mouse gonadotropin-associated peptide; inhibin; activin; vascular endothelial growth factor (VEGF); integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as, for example and not for limitation, IL-1, IL-1.alpha., IL-1.beta., IL-2, IL-3, IL-4, IL-5, IL-6, IL-7, IL-8, IL-9, IL-10, IL-11, IL-12; a tumor necrosis factor such as TNF-α or TNF-β; and other polypeptide factors including leukemia inhibitory factor (LIF) and kit ligand (KL). As used herein, when referring to a patient the term “cytokine” refers to on one or more of those produced by the patient.

The term “chemokine” is a generic term for any of the proteins that act on white blood cells and induce them to move and/or become activated to carry out their immune system functions. Chemokines are well-known in the art. Exemplary chemokines include, for example and not for limitation, TECK, ELC, BLC-1, CTACK, RANTES, fractalkine, exotaxin, eotaxin-2, Monocyte chemoattractant protein-1 (MCP-1), MCP-2, MCP-3, MCP-4, MDC, leukotactin, SDF-1.beta., lymphotactin, TARC, ITAC, ENA-70, ENA-78, IP-10, NAP-2, interleukin-8 (IL-8), HCC-1, MIP-1α, MIP-1β, MIP-1δ, I-309, GRO-α, GRO-β, GRO-γ, MPIF-1, I-LINK, and GCP-2. As used herein, when referring to a patient the term “chemokine” refers to any of those produced by the patient.

The term “agent” or “compound” as used herein refers to a chemical entity or biological product, or combination of chemical entities or biological products, administered to a subject to treat or prevent or control a disease or condition. The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G.” a thymine “T” or a cytosine “C”) or RNA (e.g. an A, a G. an uracil “U” or a C). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The term “nucleic acid” also refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The terms “polynucleotide sequence” and “nucleotide sequence” are also used interchangeably herein.

As used herein, the term “gene” refers to a nucleic acid comprising an open reading frame encoding a polypeptide, including both exon and (optionally) intron sequences. A “gene” refers to coding sequence of a gene product, as well as non-coding regions of the gene product, including 5′UTR and 3′UTR regions, introns and the promoter of the gene product. These definitions generally refer to a single-stranded molecule, but in specific embodiments will also encompass an additional strand that is partially, substantially or fully complementary to the single-stranded molecule. Thus, a nucleic acid may encompass a double-stranded molecule or a double-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “is.” The term “gene” refers to the segment of DNA involved in producing a polypeptide chain, it includes regions preceding and following the coding region as well as intervening sequences (introns) between individual coding segments (exons). A “promoter” is a region of a nucleic acid sequence at which initiation and rate of transcription are controlled. It may contain elements at which regulatory proteins and molecules may bind, such as RNA polymerase and other transcription factors, to initiate the specific transcription of a nucleic acid sequence. The term “enhancer” refers to a cis-acting regulatory sequence involved in the transcriptional activation of a nucleic acid sequence. An enhancer can function in either orientation and may be upstream or downstream of the promoter.

As used herein, the term “gene product(s)” is used to refer to include RNA transcribed from a gene (e.g., mRNA), or a polypeptide encoded by a gene or translated from RNA.

The terms “polypeptide” and “protein” are used interchangeably to refer to a polymer of amino acid residues, and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational (e.g., signal peptide cleavage of amino acids 1-20 of SEQ ID NO:1) and post-translational modifications of the polypeptide, such as, for example, disulfide-bond formation, glycosylation, acetylation, phosphorylation, proteolytic cleavage (e.g., cleavage by furins or metalloproteases and prohormone convertases (PCs)), and the like. Furthermore, for purposes of the present invention, a “polypeptide” encompasses a protein that includes modifications, such as deletions, additions, and substitutions (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, has a well-defined conformation. Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

The incorporation of non-natural amino acids, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides. In some embodiments, an Insl6.Fc are comprised of D- or L-amino acid residues, as use of naturally occurring L-amino acid residues has the advantage that any break-down products should be relatively non-toxic to the cell or organism.

In yet a further embodiment, an insl6 protein or fragments or derivatives thereof can be a retro-inverso peptides. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A. and Viscomi, G. C., J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

The term “fragment” of a peptide, polypeptide or molecule as used herein refers to any contiguous polypeptide subset of the molecule. The term “protein fragment” as used herein includes both synthetic and naturally-occurring amino acid sequences derivable from the naturally occurring amino acid sequence of Insl6 (SEQ ID NO:1). The protein is said to be “derivable from the naturally-occurring amino acid sequence of Insl6” if it can be obtained by fragmenting the naturally-occurring Insl6, or if it can be synthesized based upon a knowledge of the sequence of the naturally occurring amino acid sequence or of the genetic material (DNA or RNA) which encodes this sequence. Accordingly, a “fragment” of a molecule, is meant to refer to any polypeptide subset of the molecule. In some embodiments, a functional fragment of Insl6 comprises at least the B-domain (amino acids 21-53 of SEQ ID NO: 1) and/or the A-domain of Insl6 (amino acids 173-198 of SEQ ID NO: 1). In some embodiments, a functional fragment comprises a portion of the A-domain and/or a portion (e.g., fragment) of the B-domain of Insl6. Fragments of Insl6 which have the activity of wildtype Insl6 protein as disclosed herein and which are soluble are also encompassed for use in the present invention.

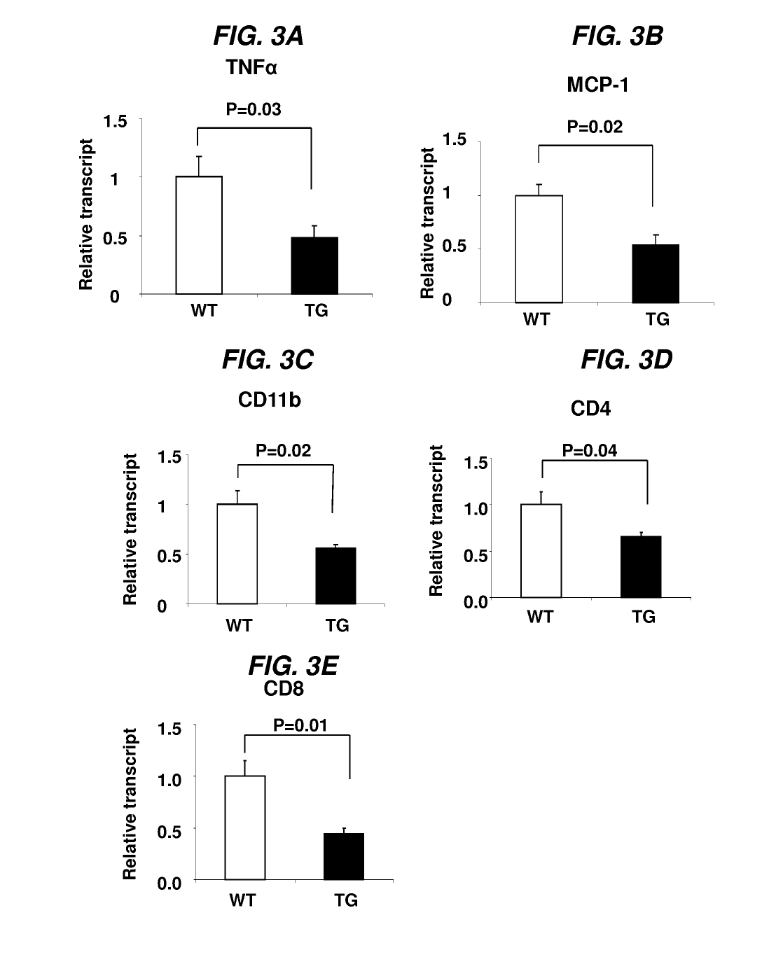

Fragments of an Insl6 protein, for example functional fragments of SEQ ID NO: 1 useful in the methods as disclosed herein have at least 30% the activity as that of a polypeptide of SEQ ID NO: 1 in vivo. Stated another way, a fragment of an Insl6 polypeptide is a fragment of any of SEQ ID NO: 1 which, alone or fused to Fc can result in at least 30% of the same activity as compared to SEQ ID NO: 1 to decrease pro-inflammatory cytokines as disclosed herein when administered to a mouse in vivo (as disclosed in the Examples and FIG. 3 herein). It can also include fragments that decrease the wild type activity of one property by at least 30%. Fragments as used herein can be soluble (i.e. not membrane bound), and typically bound to a first fusion partner, however, they do not need to be fused to a fusion protein if the Insl6 fragment is stable. A “fragment” can be at least about 6, at least about 9, at least about 15, at least about 20, at least about 30, least about 40, at least about 50, at least about 100, at least about 250, at least about 300 nucleic or amino acids, and all integers in between. Exemplary fragments include C-terminal truncations, N-terminal truncations, or truncations of both C- and N-terminals (e.g., deletions of, for example, at least 1, at least 2, at least 3, at least 4, at least 5, at least 8, at least 10, at least 15, at least 20, at least 25, at least 40, at least 50, at least 75, at least 100 or more amino acids deleted from the N-termini, the C-termini, or both). One of ordinary skill in the art can create such fragments by simple deletion analysis. Such a fragment of SEQ ID NO: 1 can be, for example, 1, 2, 3, 4, 5, 6, 7, 8, 9 or 10 amino acids or more than 10 amino acids, such as 15, 30, 50, 100 or more than 100 amino acids deleted from the N-terminal and/or C-terminal of SEQ ID NO: 1, respectively. In some embodiments, a functional fragment of Insl6 comprises the B-domain (e.g., amino acids 21-53 of SEQ ID NO:1) or a fragment of the B-domain, and/or the A-domain of Insl6 (e.g., amino acids 173-198 of SEQ ID NO:1) or a fragment of the A-domain. Persons of ordinary skill in the art can easily identify the minimal peptide fragment of SEQ ID NO: 1 useful in the methods and compositions as disclosed herein, or fusion proteins as disclosed herein, by sequentially deleting N- and/or C-terminal amino acids from SEQ ID NO: 1, or sequentially deleting N- and C-terminal amino acids from Insl6 fragments of amino acids 21-53 or 173-198, and assessing the function of the resulting peptide fragment, alone or fused to Fc. In some embodiments, a Fc fusion protein can comprise the B-domain (e.g., amino acids 21-53 of SEQ ID NO:1) or a fragment of the B-domain, and/or the A-domain of Insl6 (e.g., amino acids 173-198 of SEQ ID NO:1) or a fragment of the A-domain. One can create functional fragments with multiple smaller fragments. These can be attached by bridging peptide linkers. One can readily select linkers to maintain wild type conformation. One of ordinary skill in the art can easily assess the function of an Insl6-Fc fusion protein to decrease pro-inflammatory cytokines when administered to a CIM mouse model in vivo (as disclosed in the Examples and FIGS. 3A-3E) as compared to Insl6 corresponding to SEQ ID NO: 1 or fused to a Fc as disclosed herein. Using such an in vivo assay, if the Insl6 fragment protein has at least 30% of the biological activity of the Insl6 corresponding to SEQ ID NO: 1 as disclosed herein, then the Insl6-fragment portion of an Insl6-fragment-Fc protein is considered a valid Insl6-fragment and can used in fusion proteins and methods as disclosed herein. In some embodiments, a fragment of SEQ ID NO: 1 can be less than 200, or less than 150 or less than 100, or less than 50, or less than 20 amino acids of SEQ ID NO: 1. In some embodiments, a fragment of SEQ ID NO: 1 is less than 100 peptides in length. However, as stated above, the fragment must be at least 6 amino acids, at least about 9, at least about 15, at least about 20, at least about 30, at least about 40, at least about 50, at least about 100, at least about 250, at least about 500 nucleic acids or amino acids, or any integers in between.

The term “derivative” as used herein refers to peptides which have been chemically modified, for example but not limited to by techniques such as ubiquitination, labeling, pegylation (derivatization with polyethylene glycol) or addition of other molecules. A molecule also a “derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

The term “functional” when used in conjunction with “derivative” or “variant” or “fragment” refers to a polypeptide which possess a biological activity (either functional or structural) that is substantially similar to a biological activity of the polypeptide which it is a functional derivative, variant or functional fragment thereof. The term functional derivative is intended to include the fragments, analogues or chemical derivatives of a molecule. By “substantially similar” in this context is meant that the biological activity, e.g., transmembrane transport of associated polypeptides is at 25% or at least 35%, or at least 50% as active as a reference polypeptide, e.g., a corresponding wild-type polypeptide, and preferably at least 60% as active, 70% as active, 80% as active, 90% as active, 95% as active, 100% as active or even higher (i.e., the variant or derivative has greater activity than the wild-type), e.g., 110% as active, 120% as active, or more. Stated another way, a “substantially similar” functional fragment of Insl6 in this context is meant that at least 25%, at least 35%, at least 50% of the relevant or desired biological activity of a corresponding wild-type peptide is retained. In the instance of a functional fragment or peptide of Insl6 (e.g., SEQ ID NO: 1), a functional fragment of SEQ ID NO: 1 would be a protein or peptide comprising a portion of SEQ ID NO: 1 which retained an activity to decrease pro-inflammatory cytokines when administered to a CIM mouse model in vivo (as disclosed in the Examples and FIGS. 3A-3E); preferably the fragment of SEQ ID NO: 1 that retains at least 25%, at least 35%, at least 50% at least 60%, at least 70%, at least 80%, at least 90%, at least 95%, at least 100% or even higher (i.e., the variant or derivative has greater activity than the wild-type SEQ ID NO: 1), e.g., at least 110%, at least 120%, or more activity compared to the full length SEQ ID NO: 1 to decrease pro-inflammatory cytokines when administered to a CIM mouse model in vivo (as disclosed in the Examples and FIGS. 3A-3E). Such functional fragments can be assessed by assessing the expression level of mRNA transcripts encoding inflammatory proteins and markers, such as, but not limited to CD4, CD8, CD11b, TNF-α and MCP-1 in a mouse model of C protein-induced (poly)myositis model (CIM), in a mouse administered a functional fragment of SEQ ID NO: 1 as compared to a mouse administered a wild-type or full length Insl6 polypeptide of SEQ ID NO: 1 as disclosed herein in the Examples, where a functional fragment decreases the expression of pro-inflammatory cytokines in a CIM mouse model in vivo similar to that of wild-type of SEQ ID NO: 1 as compared to the absence of such polypeptide of SEQ ID NO: 1.

Accordingly, the term “non-functional” as used herein in conjunction with a “non-fragment of Insl6” refers to a polypeptide which comprises at least a portion of the Insl6 protein of SEQ ID NO:1 but does not retain the natural function of Insl6 of decreasing the expression of pro-inflammatory cytokines in a CIM mouse model in vivo. In some embodiments, a non-functional fragment of Insl6 still binds to the native ligands of Insl6 but does not allow Insl6-mediated intracellular signaling.

The term “functional derivative” and “mimetic” or “biologically active variant” or “biologically active fragment” are used interchangeably, and refers to a compound which possess a biological activity (either functional or structural) that is substantially similar to a biological activity of the entity or molecule its is a functional derivative of (e.g., the wildtype Insl6 protein). The term functional derivative is intended to include the fragments, variants, analogues or chemical derivatives of a molecule.

The term “functional derivatives” is intended to include the “fragments,” “variants,” “analogs,” or “chemical derivatives” of a molecule. A molecule is said to be “substantially similar” to another molecule if both molecules have substantially similar structures or if both molecules possess a similar biological activity. Thus, provided that two molecules possess a similar activity, they are considered variants as that term is used herein even if the structure of one of the molecules not found in the other, or if the sequence of amino acid residues is not identical. An “analog” of Insl6 is meant to refer to a molecule substantially similar in function to either the entire molecule or to a fragment thereof. As used herein, a molecule is said to be a “chemical derivative” of another molecule when it contains additional chemical moieties not normally a part of the molecule. Such moieties can improve the molecule's solubility, absorption, biological half life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, Pa. (1990).

A “variant” of the Insl6 protein is meant to refer to a molecule substantially similar in structure and function to either the entire molecule, or to a fragment thereof. Accordingly, the term “variant” as used herein refers to a peptide or nucleic acid that differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant when used with reference to a polynucleotide or polypeptide, refers to a polynucleotide or polypeptide that can vary in primary, secondary, or tertiary structure, as compared to a reference polynucleotide or polypeptide, respectively (e.g., as compared to a wild-type polynucleotide or polypeptide). A “variant” of Insl6 polypeptide, for example SEQ ID NO: 1 is meant to refer to a molecule substantially similar in structure and function, i.e. where the function is the ability to decrease inflammation in vivo and/or reduce autoimmune cells and/or increase Insl6 levels in a subject.

For example, a variant of an Insl6 protein can contain a mutation or modification that differs from a reference amino acid in SEQ ID NO: 1. In some embodiments, a variant of SEQ ID NO: 1 is a fragment of SEQ ID NO: 1 as disclosed herein. In some embodiments, a variant can be a different isoform of SEQ ID NO: 1 or can comprise different isomer amino acids. Variants can be naturally-occurring, synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins.

The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. “Conservative amino acid substitutions” result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. the ability of the peptide to reduce T-reg cells and/or decrease inflammatory cytokines as disclosed herein). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In some embodiments, individual substitutions, deletions or additions that alter, add or delete a single amino acid or a small percentage of amino acids can also be considered “conservative substitutions” is the change does not reduce the activity of the peptide (i.e. the ability of Insl6 protein or peptide variant to decrease pro-inflammatory cytokines in vivo). Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can select the amino acid which will substitute an existing amino acid based on the location of the existing amino acid, i.e. its exposure to solvents (i.e. if the amino acid is exposed to solvents or is present on the outer surface of the peptide or polypeptide as compared to internally localized amino acids not exposed to solvents). Selection of such conservative amino acid substitutions are well known in the art, for example as disclosed in Dordo et al, J. Mol. Biol, 1999, 217, 721-739 and Taylor et al, J. Theor. Biol. 119 (1986); 205-218 and S. French and B. Robson, J. Mol. Evol. 19 (1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In alternative embodiments, one can also select conservative amino acid substitutions encompassed suitable for amino acids on the interior of a protein or peptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants. A variant of an Insl6 protein, for example a variant of SEQ ID NO: 1 is meant to refer to any molecule substantially similar in structure and function to either the entire molecule of SEQ ID NO: 1, or to a fragment thereof.

The terms “homology”, “identity” and “similarity” refer to the degree of sequence similarity between two peptides or between two optimally aligned nucleic acid molecules. Homology and identity can each be determined by comparing a position in each sequence which can be aligned for purposes of comparison. For example, it is based upon using a standard homology software in the default position, such as BLAST, version 2.2.14. When an equivalent position in the compared sequences is occupied by the same base or amino acid, then the molecules are identical at that position; when the equivalent site occupied by similar amino acid residues (e.g., similar in steric and/or electronic nature such as, for example conservative amino acid substitutions), then the molecules can be referred to as homologous (similar) at that position. Expression as a percentage of homology/similarity or identity refers to a function of the number of similar or identical amino acids at positions shared by the compared sequences, respectfully. A sequence which is “unrelated” or “non-homologous” shares less than 40% identity, though preferably less than 25% identity with the sequences as disclosed herein.

As used herein, the term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T. C, G. U. or I) or residue occurs in both sequences to yield the number of matched positions, dividing the number of matched positions by the total number of positions in the comparison window (i.e., the window size), and multiplying the result by 100 to yield the percentage of sequence identity.

The terms “substantial identity” as used herein denotes a characteristic of a polynucleotide or amino acid sequence, wherein the polynucleotide or amino acid comprises a sequence that has at least 85% sequence identity, preferably at least 90% to 95% sequence identity, more usually at least 99% sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which can include deletions or additions which total 20 percent or less of the reference sequence over the comparison window. The reference sequence can be a subset of a larger sequence. The term “similarity”, when used to describe a polypeptide, is determined by comparing the amino acid sequence and the conserved amino acid substitutes of one polypeptide to the sequence of a second polypeptide.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides, usually from about 75% to 99%, and more preferably at least about 98 to 99% of the nucleotides. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan.

The term “substantially homologous” refers to sequences that are at least 90%, at least 95% identical, at least 96%, identical at least 97% identical, at least 98% identical or at least 99% identical. Homologous sequences can be the same functional gene in different species. Determination of homologs of the genes or peptides of the present invention can be easily ascertained by the skilled artisan.

A molecule is said to be “substantially similar” to another molecule if both molecules have substantially similar structures or if both molecules possess a similar biological activity, for example if both molecules are able to reduce pro-inflammatory cytokines in vivo. Thus, provided that two molecules possess a similar activity, (i.e. a variant of an Insl6 protein which can decrease reduce pro-inflammatory cytokines concentration in vivo similar to that of the Insl6 protein which corresponds to SEQ ID NO: 1, alone or when fused to Fc) are considered variants and are encompassed for use as disclosed herein, even if the structure of one of the molecules not found in the other, or if the sequence of amino acid residues is not identical. Thus, provided that two molecules possess a similar biological activity, they are considered variants as that term is used herein even if the structure of one of the molecules not found in the other, or if the sequence of amino acid residues is not identical. In particular, the term “substantially similar”, when used to define an Insl6 protein comprising a functional variant of Insl6 or a functional derivative of Insl6 as compared to the Insl6 protein encoded by SEQ ID NO:1, means that a particular subject sequence, for example, a Insl6 fragment or Insl6 variant or Insl6 derivative sequence, varies from the sequence of the natural (or wild-type) Insl6 protein (i.e. Insl6 encoded by SEQ ID NO: 1), by one or more substitutions, deletions, or additions, although the net effect of which is to retain at least some of the biological activity found in the native natural Insl6 protein. As such, nucleic acid and amino acid sequences having lesser degrees of similarity but comparable biological activity to Insl6 are considered to be equivalents. In determining polynucleotide sequences, all subject polynucleotide sequences capable of encoding substantially similar amino acid sequences are considered to be substantially similar to a reference polynucleotide sequence, regardless of differences in codon sequence. A nucleotide sequence is “substantially similar” to a specific nucleic acid sequence of SEQ ID NO:2 as disclosed herein if: (a) the nucleotide sequence is hybridizes to the coding regions of the natural Insl6 nucleic acid, or (b) the nucleotide sequence is capable of hybridization to nucleotide sequence of Insl6 encoded by SEQ ID NO: 2 under moderately stringent conditions and has biological activity similar to the native human Insl6 protein; or (c) the nucleotide sequences which are degenerative as a result of the genetic code to the nucleotide sequences defined in (a) or (b). Substantially similar proteins will typically be greater than about 80% similar to the corresponding sequence of the native protein.

The term “substantial similarity” in the context of polypeptide sequences, indicates that the polypeptide comprises a sequence with at least 60% sequence identity to a reference sequence, or 70%, or 80%, or 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ by one or more conservative substitutions.

In one embodiment, the term “human homolog” to a gene transcript refers to a DNA sequence that has at least about 55% homology to the full length nucleotide sequence of the sequence of the Insl6 gene as encoded by the genome of humans or an animal, for example mouse or transgenic animal. In one embodiment, the term “human homolog” to a protein identified as associated with Insl6 refers to an amino acid sequence that has 40% homology to the full length amino acid sequence of the protein identified as associated with Insl6 as encoded by the genome of the transgenic animal of the present invention, more preferably at least about 50%, still more preferably, at least about 60% homology, still more preferably, at least about 70% homology, even more preferably, at least about 75% homology, yet more preferably, at least about 80% homology, even more preferably at least about 85% homology, still more preferably, at least about 90% homology, and more preferably, at least about 95% homology. As discussed above, the homology is at least about 50% to 100% and all intervals in between (i.e., 55%, 60%, 70%, 75%, 80%, 85%, 90%, 95%, 98%, etc.). Determination of the human homologs of the genes of the present invention may be easily ascertained by the skilled artisan.

The term “conservative substitution,” when describing a polypeptide, refers to a change in the amino acid composition of the polypeptide that does not substantially alter the polypeptide's activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.”

As used herein, the term “nonconservative” refers to substituting an amino acid residue for a different amino acid residue that has different chemical properties. The nonconservative substitutions include, but are not limited to aspartic acid (D) being replaced with glycine (G); asparagine (N) being replaced with lysine (K); or alanine (A) being replaced with arginine (R).

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins and Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

The term “insertions” or “deletions” are typically in the range of about 1 to 5 amino acids. The variation allowed can be experimentally determined by producing the peptide synthetically while systematically making insertions, deletions, or substitutions of nucleotides in the sequence using recombinant DNA techniques.

The term “substitution” when referring to a peptide, refers to a change in an amino acid for a different entity, for example another amino acid or amino-acid moiety. Substitutions can be conservative or non-conservative substitutions.

An “analog” of a molecule such as Insl6 peptide, for example SEQ ID NO: 1 refers to a molecule similar in function to either the entire molecule or to a fragment thereof. The term “analog” is also intended to include allelic, species and induced variants. Analogs typically differ from naturally occurring peptides at one or a few positions, often by virtue of conservative substitutions. Analogs typically exhibit at least 80 or 90% sequence identity with natural peptides. Some analogs also include unnatural amino acids or modifications of N or C terminal amino acids. Examples of unnatural amino acids are, for example but not limited to; acedisubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine. Fragments and analogs can be screened for prophylactic or therapeutic efficacy in transgenic animal models as described below.

By “covalently bonded” is meant joined either directly or indirectly (e.g., through a linker) by a covalent chemical bond.

The term “fusion protein” as used herein refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. As a non-limiting example, the nucleic acid sequence encoding the Insl6 fusion protein is derived from the nucleotide sequence of encoding a Insl6 protein or a functional derivative fragment or variant thereof, fused in frame to an end, either the 5′ or the 3′ end, of a gene encoding a first fusion partner, such as a IgG1 Fc fragment. In this manner, on expression of the gene, the Insl6 protein or a functional derivative fragment or variant thereof is functionally expressed and fused to the N-terminal or C-terminal end of the IgG1 Fc. In certain embodiments, modification of the polypeptide probe is such that the functionality of the Insl6 protein or a functional derivative fragment or variant thereof remains substantially unaffected in terms of its biological activity by fusion to the first fusion partner, such as IgG1 Fc.

By “specifically binds” or “specific binding” is meant a compound or antibody that recognizes and binds a desired polypeptide but that does not substantially recognize and bind other molecules in a sample, for example, a biological sample, which naturally includes a polypeptide of the invention.

By “substantially pure” or is meant a nucleic acid, polypeptide, or other molecule that has been separated from the components that naturally accompany it. Typically, a polypeptide is substantially pure when it is at least about 60%, or at least about 70%, at least about 80%, at least about 90%, at least about 95%, or even at least about 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

By “enhanced proteolytic stability” is meant a reduction of in the rate or extent of proteolysis of a peptide sequence by at least about 2%, at least about 5%, at least about 10%, at least about 20%, at least about 30%, at least about 40%, at least about 50%, at least about 60%, at least about 70%, at least about 80%, at least about 85%, at least about 90%, at least about 95%, at least about 98%, or at least about 99% as compared to a control sequence under the same conditions (e.g., in vivo or in an in vitro system such as in a cell or cell lysate). A peptide with enhanced proteolytic stability may contain any modification, for example, insertions, deletions, or point mutations which reduce or eliminate a site subject to proteolytic cleavage at a particular site. Sites of proteolytic cleavage may be identified based on known target sequences or using computer software (e.g., software described by Gasteiger et al., Protein Identification and Analysis Tools on the ExPASy Server. In John M. Walker, ed. The Proteomics Protocols Handbook, Humana Press (2005)). Alternatively, proteolytic sites can be determined experimentally, for example, by Western blot for the protein following expression or incubation in a cellular system or cellular lysate, followed by sequencing of the identified fragments to determine cleavage sites.

The term “recombinant” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide with which it is associated in nature. The term recombinant as used with respect to a protein or polypeptide, means a polypeptide produced by expression of a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “subject” and “individual” are used interchangeably herein, and refer to an animal, for example a human, to whom treatment, including prophylactic treatment, with the pharmaceutical composition according to the present invention, is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. The term does not denote a particular age or sex. Thus, adult and newborn subjects, as well as fetuses, whether male or female, are intended to be covered. Examples of subjects include humans, dogs, cats, cows, goats, and mice. The term subject is further intended to include transgenic species.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, amlady, disorder, sickness, illness, complaint, inderdisposion, affection.

A “composition” or “pharmaceutical composition” are used interchangeably herein refers to a composition that usually contains an excipient, such as a pharmaceutically acceptable carrier that is conventional in the art and that is suitable for administration to cells. The cells may be part of a subject, for example for therapeutic, diagnostic, or prophylactic purposes. The cells may also be cultured, for example cells as part of an assay for screening potential pharmaceutical compositions, and the cells may be part of a transgenic animal for research purposes. The composition can also be a cell culture, in which a polypeptide or polynucleotide encoding a metabolic regulator of the present invention is present in the cells and/or in the culture medium. In addition, compositions for topical (e.g., oral mucosa, respiratory mucosa) and/or oral administration can form solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

As used herein, the terms “treat,” “treating,” and “treatment” refer to the alleviation or measurable lessening of one or more symptoms or measurable markers of a disease or disorder; while not intending to be limited to such, disease or disorders of particular interest include autoimmune diseases and myositis. Measurable lessening includes any statistically significant decline in a measurable marker or symptom. In some embodiments, treatment is prophylactic treatment.

The term “therapeutically effective amount” refers to an amount effective, at dosages and for periods of time necessary, to achieve the desired therapeutic result, e.g., a diminishment or prevention of effects associated with various disease states or conditions, such as reduce a symptom of an autoimmune disease in the subject. The term “therapeutically effective amount” refers to an amount of an Insl6 agent as disclosed herein (e.g., an Insl6 protein, or fragment or fusion protein thereof, or an nucleic acid Insl6 agent encoding an Inls6 protein, or fragment, derivative or fusion protein thereof, with one or more therapeutic agents) effective to treat or prevent a disease or disorder in a mammal, preferably a human. In the case of treatment of an autoimmune and/or inflammatory disease, a therapeutically effective amount may alleviate one or more symptoms associated with the disease including decreasing or stabilizing pain, swelling, discomfort and/or tissue damage. For rheumatoid arthritis, efficacy and response may represent achieving the American College of Rheumatology ACR20 or ACR50 scores. In Crohn's disease, therapeutically effective doses may lower the Disease Activity Index (CDAI). This effect may be achieved by reducing circulating levels of pro-inflammatory cytokines and chemokines and/or decreasing the numbers of inflammatory infiltrating cells such as monocytes and macrophages. To the extent that the drug or drug composition may affect effector cell (monocyte, macrophage, etc.) function or activity, it may block or interfere with the activity of these cells (e.g., reduce cytokine production) or it may kill existing cells by inducing their apoptosis. A therapeutically effective amount of an Insl6 agent, e.g., Insl6 protein or functional fragment or variant thereof can vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects. In some embodiments, a therapeutically effective amount is an ‘effective amount”, which as used herein refers to the amount of therapeutic agent of pharmaceutical composition to alleviate at least one or some of the symptoms of the disease or disorder. An “effective amount” for purposes herein is thus determined by such considerations as are known in the art and is the amount to achieve improvement including, but not limited to, improved survival rate or more rapid recovery, or improvement or elimination of at least one symptom and other indicator of an autoimmune disease which are appropriate measures by those skilled in the art. It should be noted that Insl6 agents as disclosed herein can be administered as a pharmaceutically acceptable salt and can be administered alone or as an active ingredient in combination with pharmaceutically acceptable carriers, diluents, adjuvants and vehicles. In some embodiments, the effective amount is a reduction or decrease in the level, by at least 10% , of at least one inflammatory cytokine in the blood or muscle biopsy in a biological sample obtained from the subject (e.g., an inflammatory cytokine from, but not limited to, the group of CD4, CD8, CD11b, TNFα and MCP-1). In some embodiments, an effective amount is the amount of an Insl6 agent that is effective at reducing the level or number of T-reg cells by at least 10% in a biological sample, e.g., a blood or muscle biopsy sample, obtained from the subject.

The term “prophylactically effective amount” refers to an amount of an Insl6 agent, e.g., Insl6 protein or functional fragment or variant thereof which is effective, at dosages and for periods of time necessary, to achieve the desired prophylactic result, e.g., to prevent the onset of an autoimmune disease where the subject is at risk of developing an autoimmune disease. Typically, since a prophylactic dose of an Insl6 agent, e.g., Insl6 protein or functional fragment or variant thereof is administered to a subject prior to or at an earlier stage of an autoimmune disease, and in some embodiments, a prophylactically effective amount is less than the therapeutically effective amount. A prophylatically effective amount of an Insl6 agent, e.g., Insl6 protein or functional fragment or variant thereof is also one in which any toxic or detrimental effects of the compound are outweighed by the beneficial effects.

As used herein, the terms “prevent,” “preventing” and “prevention” refer to the avoidance or delay in manifestation of one or more symptoms or measurable markers of a disease or disorder, e.g., of an autoimmune disease. A delay in the manifestation of a symptom or marker is a delay relative to the time at which such symptom or marker manifests in a control or untreated subject with a similar likelihood or susceptibility of developing the disease or disorder. The terms “prevent,” “preventing” and “prevention” include not only the avoidance or prevention of a symptom or marker of the disease, but also a reduced severity or degree of any one of the symptoms or markers of the disease, relative to those symptoms or markers in a control or non-treated individual with a similar likelihood or susceptibility of developing the disease or disorder, or relative to symptoms or markers likely to arise based on historical or statistical measures of populations affected by the disease or disorder. By “reduced severity” is meant at least a 10% reduction in the severity or degree of a symptom or measurable disease marker, relative to a control or reference, e.g., at least 15%, 20%, 30%, 40%, 50%, 60%, 70%, 80%, 90%, 95%, 99% or even 100% (i.e., no symptoms or measurable markers).

As used herein, the terms “administering,” and “introducing” are used interchangeably herein and refer to the placement of the agents of metabolic regulators of the present invention into a subject by a method or route which results in at least partial localization of the Insl6 agents at a desired site. The compounds of the present invention can be administered by any appropriate route which results in an effective treatment in the subject.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of Insl6 agent such that it enters the animal's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The term “regeneration” means regrowth of a cell population, organ or tissue, and in some embodiments after disease or trauma.

The term “vectors” refers to a nucleic acid molecule capable of transporting another nucleic acid to which it has been linked; a plasmid is a species of the genus encompassed by “vector”. The term “vector” typically refers to a nucleic acid sequence containing an origin of replication and other entities necessary for replication and/or maintenance in a host cell. Vectors capable of directing the expression of genes and/or nucleic acid sequence to which they are operatively linked are referred to herein as “expression vectors”. In general, expression vectors of utility are often in the form of “plasmids” which refer to circular double stranded DNA loops which, in their vector form are not bound to the chromosome, and typically comprise entities for stable or transient expression or the encoded DNA. Other expression vectors can be used in the methods as disclosed herein for example, but are not limited to, plasmids, episomes, bacterial artificial chromosomes, yeast artificial chromosomes, bacteriophages or viral vectors, and such vectors can integrate into the host's genome or replicate autonomously in the particular cell. A vector can be a DNA or RNA vector. Other forms of expression vectors known by those skilled in the art which serve the equivalent functions can also be used, for example self replicating extrachromosomal vectors or vectors which integrates into a host genome. Preferred vectors are those capable of autonomous replication and/or expression of nucleic acids to which they are linked Vectors capable of directing the expression of genes to which they are operatively linked are referred to herein as “expression vectors”.

The term “viral vectors” refers to the use as viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. Enhancers need not be located in close proximity to the coding sequences whose transcription they enhance. Furthermore, a gene transcribed from a promoter regulated in trans by a factor transcribed by a second promoter may be said to be operatively linked to the second promoter. In such a case, transcription of the first gene is said to be operatively linked to the first promoter and is also said to be operatively linked to the second promoter.

As used herein, the term “biological sample” also refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from a subject, but the term can also refer to non-cellular biological material, such as non-cellular fractions of blood, saliva, or urine, that can be used to measure protein phosphorylation levels. In some embodiments, a “biological sample” or “tissue sample” refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, a biological sample is from a resection, bronchoscopic biopsy, or core needle biopsy of a primary, secondary or metastatic tumor, or a cellblock from pleural fluid. In addition, fine needle aspirate biological samples are also useful. In some embodiments, a biological sample is primary ascite cells. Samples can be fresh, frozen, fixed or optionally paraffin-embedded, frozen or subjected to other tissue preservation methods, including for example methods to preserve the phosphorylation status of polypeptides in the biological sample. A biological sample can also mean a sample of biological tissue or fluid that comprises protein or cells. Such samples include, but are not limited to, tissue isolated from subjects or animals. Biological samples may also include sections of tissues such as biopsy and autopsy samples, frozen sections taken for histological purposes, blood, plasma, serum, sputum, stool, tears, mucus, hair, and skin. Biological samples also include explants and primary and/or transformed cell cultures derived from patient tissues. A biological sample may be provided by removing a sample of cells from subject, but can also be accomplished by using previously isolated cells (e.g., isolated by another person, at another time, and/or for another purpose), or by performing the methods of the invention in vivo. Archival tissues, such as those having treatment or outcome history may also be used. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. The biological sample can be obtained by removing a sample of cells from a subject, but can also be accomplished by using previously isolated cells (e.g. isolated by another person), or by performing the methods of the invention in vivo. Such samples include, but are not limited to, whole blood, cultured cells, primary cell preparations, sputum, amniotic fluid, tissue or fine needle biopsy samples, peritoneal fluid, and pleural fluid, among others. In some embodiments a biological sample is taken from a human patient, and in alternative embodiments the biological sample is taken from any mammal, such as rodents, animal models of diseases, commercial animals, companion animals, dogs, cats, sheep, cattle, and pigs, etc. The biological sample can be pretreated as necessary for storage or preservation, by dilution in an appropriate buffer solution or concentrated, if desired. Any of a number of standard aqueous buffer solutions, employing one of a variety of buffers, such as phosphate, Tris, or the like, at physiological pH can be used. The biological sample can in certain circumstances be stored for use prior to use in the assay as disclosed herein. Such storage can be at +4 C or frozen, for example at −20 C or −80 C, provided suitable cryopreservation agents are used to maintain cell viability once the cells are thawed.

The term “reduced” or “reduce” or “decrease” as used herein generally means a decrease by a statistically significant amount relative to a reference. However, for avoidance of doubt, “reduced” means statistically significant decrease of at least 10% as compared to a reference level, for example a decrease by at least 20%, at least 30%, at least 40%, at least t 50%, or least 60%, or least 70%, or least 80%, at least 90% or more, up to and including a 100% decrease (i.e. absent level as compared to a reference sample), or any decrease between 10-100% as compared to a reference level, as that term is defined herein. The term “decrease” or “inhibition” used in the context of the level of expression or activity of a gene refers to a reduction in protein or nucleic acid level or activity in a cell, a cell extract, or a cell supernatant. For example, such a decrease may be due to reduced RNA stability, transcription, or translation, increased protein degradation, or RNA interference. Preferably, this decrease is at least about 5%, at least about 10%, at least about 25%, at least about 50%, at least about 75%, at least about 80%, or even at least about 90% of the level of expression or activity under control conditions.

The term “low” as used herein generally means lower by a statically significant amount; for the avoidance of doubt, “low” means a statistically significant value at least 10% lower than a reference level, for example a value at least 20% lower than a reference level, at least 30% lower than a reference level, at least 40% lower than a reference level, at least 50% lower than a reference level, at least 60% lower than a reference level, at least 70% lower than a reference level, at least 80% lower than a reference level, at least 90% lower than a reference level, up to and including 100% lower than a reference level (i.e. absent level as compared to a reference sample).

The terms “increased” or “increase” as used herein generally mean an increase by a statically significant amount; for the avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein. The term an “increase” as used in the context of the expression or activity of a gene or protein is meant a positive change in protein or nucleic acid level or activity in a cell, a cell extract, or a cell supernatant. For example, such a increase may be due to increased RNA stability, transcription, or translation, or decreased protein degradation. Preferably, this increase is at least 5%, at least about 10%, at least about 25%, at least about 50%, at least about 75%, at least about 80%, at least about 100%, at least about 200%, or even about 500% or more over the level of expression or activity under control conditions.

The term “high” as used herein generally means a higher by a statically significant amount relative to a reference; for the avoidance of doubt, “high” means a statistically significant value at least 10% higher than a reference level, for example at least 20% higher, at least 30% higher, at least 40% higher, at least 50% higher, at least 60% higher, at least 70% higher, at least 80% higher, at least 90% higher, at least 100% higher, at least 2-fold higher, at least 3-fold higher, at least 4-fold higher, at least 5-fold higher, at least 10-fold higher or more, as compared to a reference level.

The articles “a” and an are used herein to refer to one or to more than one (i.e., to at least one) of the grammatical object of the article. By way of example, “an element” means one element or more than one element.

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the following Detailed Description, the drawings, and the claims.

### Insulin-Like 6 (Insl6) Agents

Without wishing to be bound by theory, the Insulin-like protein 6 (Insl6) is a member of the relaxin family, and is identified as RefSeq ID: NM_{—}007179 (SEQ ID NO:2) or the amino acid sequence NP_{—}009110 (SEQ ID NO:1). Insl6 is also known by aliases Relaxin, insulin-like factor 1, insulin like peptide Insl6 B chain, insulin-like peptide Insl6 A chain. In some embodiments, Insl6 is the human homologue of Insl6 or a human cognate of Insl6, and in some embodiments, Insl6 is a rodent isoform of Insl6, or any mammalian or non-mammalian animal isoform of Insl6, for example invertebrate Insl6 or primate Insl6. Also encompassed by the term Insl6 are all variants and homologues of Insl6, and functional derivatives of Insl6, for example mutant variants and/or alternative isoforms of Insl6, for example alternative spliced isoforms of Insl6, or fragments of Insl6 such as, for example Insl6 lacking the signal peptide, or recombinant forms of Insl6.

In some embodiments, insl6 polypeptide for use in the methods and compositions as disclosed herein can be a prohormone comprising a signal sequence, a B-, C-, and an A-chain domain (e.g., SEQ ID NO:1). Processing of the mature hormone Insl6 can involve the proteolytic cleavage and removal of the signal peptide (e.g., amino acids 1-20 of SEQ ID NO: 1), the joining of the B-domain (amino acids 21-53 of SEQ ID NO:1) and A-domain (amino acids 173-198 of SEQ ID NO:1) by inter- and intrachain disulfide bonds, and, in some embodiments, the proteolytic removal of the connecting C-domain peptide (amino acids 55-168 of SEQ ID NO: 1) by a prohormone convertase. In some embodiments, the propeptide fragment of SEQ ID NO: 1 (e.g., amino acids 201-213 of SEQ ID NO:1) is also removed from the Insl6 polypeptide prior to use in the methods and compositions as disclosed herein.

Accordingly, in some embodiments, an insl6 polypeptide for use in the methods and compositions as disclosed herein can comprise at least amino acids 21-53 and 173-198 of SEQ ID NO: 1, which can be conjugated, e.g., by a peptide bond or joined by a disulfide or other peptide bond. In some embodiments, a nucleic acid or modified RNA (modRNA) encoding an insl6 polypeptide comprising least amino acids 21-53 and 173-198 of SEQ ID NO: 1 is encompassed for use in the methods and compositions as disclosed herein. In some embodiments, an Insl6 polypeptide useful in the methods and compositions as disclosed herein comprises functional fragments of amino acids 21-53 and 173-198 of SEQ ID NO: 1.

In some embodiments, an insl6 agent is a protein or polypeptide of Insl6. In some embodiments, an insl6 agent can be a modified RNA (mod-RNA) encoding the Insl6 protein. Modified RNA is commonly known by persons of ordinary skill in the art and disclosed in patent applications US 2012/0046346, WO 2012/078637, WO2012/019168, WO2012/045075, WO2012/158736, WO2012/045082, US2012/031781, US2012/0251618, which are all incorporated herein in their entirety by reference. In some embodiments, the insl6 agent can be an activating RNAi agent (RNAa), as that term is disclosed herein.

Where an insl6 agent is an Insl6 protein, the insl6 protein, or functional fragment thereof can be a “homologous” or “heterologous polypeptide.” A “heterologous polypeptide,” also referred to as a “xenogenic polypeptide,” is a polypeptide having an amino acid sequence found in an organism not consisting of the transgenic non-human animal. As used herein, the term “polypeptide” refers to a polymer of amino acids and its equivalent and does not refer to a specific length of the product; thus, peptides, oligopeptides and proteins are included within the definition of a polypeptide. A derivative is a polypeptide having conservative amino acid substitutions, as compared with another sequence. Derivatives further include other modifications of proteins, including, for example, modifications such as glycosylations, acetylations, phosphorylations, and the like.

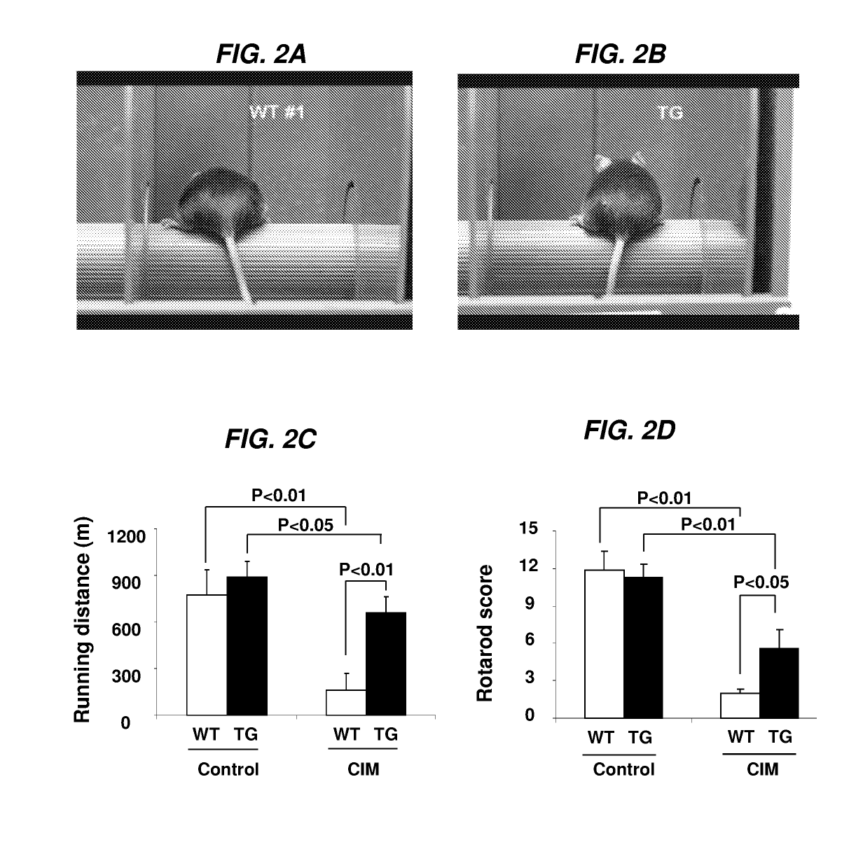

Insl6 genes containing various gene segments encoding a cognate heterologous protein sequence may be readily identified, e.g. by hybridization or DNA sequencing, as being from a species of organism other than the transgenic animal. In some embodiments, a gene sequence encoding a cognate Insl6 heterologous protein is at least 75%, at least 80%, at least 85%, at least 90% or at least 95% identical to the homologous Insl6 transgene. As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection. The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide. Multiple alignment of insulin gene family members with human and rat INSL6 over the B- and A-domains shows Insl6 has highly conserved A- and B-domain motifs, as disclosed in FIG. 2, in Lok et al., Biology of Reproduction, 2000; 62(6); 1593-1599, which is incorporated herein in its entirety by reference. The A-domain motif of Insl6 is CC X3 C X8 C, where XN represents the number of residues comprising any amino acids other than cysteine. The B-domain motif of Insl6 is LCG X10 C. Within the B- and A-domains, human INSL6 was 55% identical to rat INSL6, 43% identical to human relaxin H2, 38% identical to human Leydig INSL3, 36% identical to human insulin, 36% identical to human IGF-II, 33% identical to human IGF-I, 28% identical to human INSL5, and 24% identical to human placenta INSL4. Human and rat INSL6 C-domain sequences had no significant sequence similarity with the C-domain sequences of the other family members, but exhibited a significant degree of similarity (43%) with each other.

In some embodiments, an Insl6 protein is at least 75%, at least 80%, at least 85%, at least 90% or at least 95% similar to the homologous (e.g., wildtype) Insl6 protein. As used herein, “similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

Homologues and functional derivatives and functional fragments of Insl6 of SEQ ID NO: 1 are also encompassed for use in the present invention, and can also be identified, for example, by expression of Insl6 from an expression library. (See, e.g., Sambrook et al. (2001). Molecular cloning: a laboratory manual, 3rd ed. (Cold Spring Harbor, N.Y., Cold Spring Harbor Laboratory Press); Ausubel et al., supra.) A mutated endogenous gene sequence can be referred to as a heterologous transgene; for example, a transgene encoding a mutation in Insl6 which is not known in naturally-occurring genomes is a heterologous transgene with respect to murine and non-murine, e.g., human species. A Insl6 protein, such as, for example, those disclosed in U.S. Patent Publication Nos. 2004/0048255 and 2004/0132156 (the disclosures of which are incorporated by reference herein).

Polypeptides of Insl6 of the present invention, functional fragments and derivatives thereof can be obtained by any suitable method. For example, polypeptides can be produced using conventional recombinant nucleic acid technology such as DNA or RNA, preferably DNA. Guidance and information concerning methods and materials for production of polypeptides using recombinant DNA technology can be found in numerous treatises and reference manuals. See, e.g., Sambrook et al, 1989, Molecular Cloning—A Laboratory Manual, 2nd Ed., Cold Spring Harbor Press; Ausubel et al. (eds.), 1994, Current Protocols in Molecular Biology, John Wiley & Sons, Inc.; Innis et al. (eds.), 1990 PCR Protocols, Academic Press.

Alternatively, Insl6 polypeptides or functional fragments thereof can be obtained directly by chemical synthesis, e.g., using a commercial peptide synthesizer according to vendor's instructions. Methods and materials for chemical synthesis of polypeptides are well known in the art. See, e.g., Merrifield, 1963, “Solid Phase Synthesis,” J. Am. Chem. Soc. 83:2149-2154.

Insl6 polypeptide can be introduced into a cell using conventional techniques for transporting proteins into intact cells, e.g., by fusing the polypeptide to the internalization peptide sequence derived from Antennapedia (Bonfanti et al., Cancer Res. 57:1442-1446) or to a nuclear localization protein such as HIV tat peptide (U.S. Pat. No. 5,652,122).

Alternatively, an Insl6 polypeptide, or functional fragment or derivative or variant thereof can be expressed in the cell following introduction of a DNA encoding the protein, e.g., a nucleic acid encoding Insl6 or homologues or functional derivatives thereof, e.g., in a conventional expression vector or by a catheter or by cells transformed with the nucleic acid ex vivo and transplanted into the subject.

In some embodiments, agents that are Insl6 polypeptides or peptides, or functional fragments or homologues or variants thereof are cleavable peptides. Cleavable peptide is a peptide comprising an amino acid sequence that is recognized by a protease or peptidase or other cleaving agent expressed by a cell and found in surrounding tissue, or produced by a microbe capable of establishing an infection in a mammal. Enzyme-cleavable peptides can, but are not required to, contain one or more amino acids in addition to the amino acid recognition sequence; additional amino acids can be added to the amino terminal, carboxy terminal, or both the amino and carboxy terminal ends of the recognition sequence. Means of adding amino, acids to an amino acid sequence, e.g., in an automated peptide synthesizer, as well as means of detecting cleavage of a peptide, e.g., by chromatographic analysis for the amino acid products of such cleavage, are well known to ordinarily skilled artisans given the teachings of this invention. Enzyme-cleavable peptides, typically from about 2 to 20 amino acids in length.

The peptide or polypeptide agent useful in the present invention can be modified at their amino termini, for example, so as to increase their hydrophilicity. Increased hydrophobicity enhances exposure of the peptides on the surfaces of lipid-based carriers into which the parent peptide-lipid conjugates have been incorporated. Polar groups suitable for attachment to peptides so as to increase their hydrophilicity are well known, and include, for example and without limitation: acetyl (“Ac”), 3-cyclohexylalanyl (“Cha”), acetyl-serine (“Ac Ser”), acetyl-seryl-serine (“Ac-Ser-Ser-”), succinyl (“Suc”), succinyl-serine (“Suc-Ser”), succinyl-seryl-serine (“Suc-Ser-Ser”), methoxy succinyl (“MeO-Suc”), methoxy succinyl-serine (“MeO-Suc-Ser”), methoxy succinyl-seryl-serine (“MeO-Suc-Ser-Ser”) and seryl-serine (“Ser-Ser-”) groups, polyethylene glycol (“PEG”), polyacrylamide, polyacrylomorpholine, polyvinylpyrrolidine, a polyhydroxyl group and carboxy sugars, e.g., lactobionic, N-acetyl neuraminic and sialic acids, groups. The carboxy groups of these sugars would be linked to the N-terminus of the peptide via an amide linkage. Presently, the preferred N-terminal modification is a methoxy-succinyl modification.

Many members of the insulin family undergo post-translational modification to produce the biologically active hormone. Thus, in the prototypic example of insulin the signal peptide is proteolytically cleaved, the C-peptide is excised by prohormone convertases (PC1/PC3 and PC2), and the B-domain (amino acids 21-53 of SEQ ID NO:1) and A-domain (amino acids 173-198 of SEQ ID NO:1) are linked by inter- and intra-disulfide bonds to produce the biologically active form of Insl6. Prohormone protein convertases constitute a family of serine proteases structurally related to bacterial subtilisins and to yeast kexin. Several eukaryotic members of this family are currently known. Prohormone Convertases (PC's) cleave precursor polypeptides at specific basic residues, most often after selected paired basic residues, to generate bioactive peptide and proteins. Many members of the insulin family of proteins (e.g. Insulin, Igf-1) are substrates for PC's. In some embodiments, an Insl6 protein can be cleaved into fragment proteins which represent B-C-A (e.g., amino acids 21-198 of SEQ ID NO; 1), C-A (amino acids 55-198 of SEQ ID NO:1), and A peptides (amino acids 173-198 of SEQ ID NO:1) respectively, suggesting that analogous to insulin, Insl6 also undergoes post-translational processing. Accordingly, in some embodiments, a fragment of Insl6, e.g., a functional fragment or biologically active fragment of Insl6, for use in the composition and methods as disclosed herein comprises at least the “A” chain of Insl6 (amino acids 173-198 of SEQ ID NO:1), and/or at least the “B” chain of Insl6 (amino acids 21-53 of SEQ ID NO:1). In some embodiments a functional fragment of Insl6 comprises a functional fragment of the A- and/or B-chains of Insl6.

Methods known in the art for the therapeutic delivery of Insl6 agents such as proteins and/or nucleic acids can be used for the delivery of an agent polypeptide or an agent nucleic acid encoding a metabolic regulator of the present invention for modulating a metabolic function in a subject, e.g., cellular transfection, gene therapy, direct administration with a delivery vehicle or pharmaceutically acceptable carrier, indirect delivery by providing recombinant cells comprising a nucleic acid encoding a targeting fusion polypeptide of the invention.

Various delivery systems are known and can be used to administer an Insl6 polypeptide to a subject, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, pulmonary, intranasal, intraocular, epidural, and oral routes. The Insl6 polypeptides can be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions comprising an Insl6 polypeptide into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It will be appreciated by those of skill that an Insl6 protein or their functional fragments, variants or homologues can be readily manipulated to alter the amino acid sequence of a protein. A genes encoding the Insl6 protein or a functional fragment, homologue or variant thereof, can be manipulated by a variety of well known techniques for in vitro mutagenesis, among others, to produce variants of the naturally occurring human protein or fragment thereof, herein referred to as variants or muteins, may be used in accordance with the invention.

The variation in primary structure of an Insl6 protein, or functional fragment, or a homologue are encompassed for use in the present invention, for instance, may include deletions, additions and substitutions. The substitutions may be conservative or non-conservative. The differences between the natural Insl6 protein (e.g., wild-type protein) and a variant generally conserve desired properties, mitigate or eliminate undesired properties and add desired or new properties. For example, variants of an insl6 protein can have superior activity as compared to wild-type Insl6 protein and this functions as agonists. In some embodiments, where it is desirable to inhibit Insl6 activity, a variant with decreased activity or has the opposite function as compared to the wild type Insl6 protein can function as an antagonist, for example, but not limited to a dominant negative form of a metabolic regulator.

### Insl6-Fc Fusion Proteins

An Insl6 protein may be fused to one or more fusion partners. In certain embodiments, one of the fusion partners is the Fc protein (e.g., mouse Fc or human Fc). The fusion protein may further include a second fusion partner such as a purification or detection tag, for example, proteins that may be detected directly or indirectly such as green fluorescent protein, hemagglutinin, or alkaline phosphatase), DNA binding domains (for example, GAL4 or LexA), gene activation domains (for example, GAL4 or VP16), purification tags, or secretion signal peptides (e.g., preprotyrypsin signal sequence). In other embodiments the fusion partner may be a tag, such as c-myc, poly histidine, or FLAG. Each fusion partner may contain one or more domains, e.g., a preprotrypsin signal sequence and FLAG tag.

In one embodiment, an Insl6 fusion protein useful in the methods and compositions as disclosed herein can comprise a human Fc protein or a functional fragment thereof. Accordingly, in one embodiment, an Insl6 fusion protein useful in the methods and compositions as disclosed herein can comprises a human Fc molecule as the first fusion partner, where the Fc fragment can be SEQ ID NO: 3 or functional variants or functional derivatives thereof, where SEQ ID NO: 3 is as follows:

LELVPRGSGDPIEGRGGGGGDPKSCDKPHTCPLCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KATPPVLDSDGSFFLYSKLTVDKSRWQQGNVFSCSVMHEALHNHYTQKS
LSLSPGK

Variations and modifications to an Insl6 protein and vectors can be used to increase or decrease Insl6 fusion protein expression, and to provide means for targeting. For example, an Insl6 fusion protein can be linked with a molecular targeting molecule for muscle cells, to make the Insl6 fusion proteins tissue specific.

In one embodiment, an Insl6 fusion protein is fused to a second fusion partner, such as a carrier molecule to enhance its bioavailability. Such carriers are known in the art and include poly (alkyl) glycol such as poly ethylene glycol (PEG). Fusion to serum albumin can also increase the serum half-life of therapeutic polypeptides.

In some embodiments, an Insl6 fusion polypeptide can also be fused to a second fusion partner, for example, to a polypeptide that targets the product to a desired location, or, for example, a tag that facilitates its purification, if so desired. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use.

In some embodiments, the Insl6 protein or functional fragment thereof is modified to achieve adequate circulating half-lives, which impact dosing, drug administration and efficacy. Many approaches have been undertaken with the aim to increase the half-life of biotherapeutics. Small proteins below 60 kD are cleared rapidly by the kidney and therefore do not reach their target. This means that high doses are needed to reach efficacy. The modifications to Insl6 proteins and fragments encompassed in the methods of the present invention to increase the half-life of proteins in circulation include: PEGylation; conjugation or genetic fusion with proteins, e.g., transferrin (WO06096515A2), albumin, growth hormone (US2003104578AA); conjugation with cellulose (Levy and Shoseyov, 2002); conjugation or fusion with Fc fragments; glycosylation and mutagenesis approaches (Carter, 2006), which are incorporated herein by reference.

In the case of PEGylation, polyethylene glycol (PEG) is conjugated to an Insl6 protein and fragment, which can be for example a plasma protein, antibody or antibody fragment. The first studies regarding the effect of PEGylation of antibodies were performed in the 1980s. The conjugation can be done either enzymatically or chemically and is well established in the art (Chapman, 2002; Veronese and Pasut, 2005). With PEGylation the total size can be increased, which reduces the chance of renal filtration. PEGylation further protects from proteolytic degradation and slows the clearance from the blood. Further, it has been reported that PEGylation can reduce immunogenicity and increase solubility. The improved pharmacokinetics by the addition of PEG is due to several different mechanisms: increase in size of the molecule, protection from proteolysis, reduced antigenicity, and the masking of specific sequences from cellular receptors. In the case of antibody fragments (Fab), a 20-fold increase in plasma half-life has been achieved by PEGylation (Chapman, 2002).

To date there are several approved PEGylated drugs, e.g., PEG-interferon alpha2b (PEG-INTRON) marketed in 2000 and alpha2a (Pegasys) marketed in 2002. A PEGylated antibody fragment against TNF alpha, called Cimzia or Certolizumab Pegol, was filed for FDA approval for the treatment of Crohn's disease in 2007 and has been approved on Apr. 22, 2008. A limitation of PEGylation is the difficulty in synthesizing long monodisperse species, especially when PEG chains over 1000 kD are needed. For many applications, polydisperse PEG with a chain length over 10000 kD is used, resulting in a population of conjugates having different length PEG chains, which need extensive analytics to ensure equivalent batches between productions. The different length of the PEG chains may result in different biological activities and therefore different pharmacokinetics. Another limitation of PEGylation is a decrease in affinity or activity as it has been observed with alpha-interferon Pegasys, which has only 7% of the antiviral activity of the native protein, but has improved pharmacokinetics due to the enhanced plasma half-life.

In some embodiments, an Insl6 protein or fragment thereof is conjugated with a long lived protein, e.g. albumin, which is 67 kD and has plasma half-life of 19 days in human (Dennis et al., 2002). Albumin is the most abundant protein in plasma and is involved in plasma pH regulation, but also serves as a carrier of substances in plasma. In the case of CD4, increased plasma half-life has been achieved after fusing it to human serum albumin (Yeh et al., 1992). Other examples for fusion proteins are insulin, human growth hormone, transferrin and cytokines (Ali et al., 1999; Duttaroy et al., 2005; Melder et al., 2005; Osborn et al., 2002a; Osborn et al., 2002b; Sung et al., 2003) and see (US2003104578A1, WO06096515A2, and WO07047504A2, herein incorporated in entirety by reference).

The effect of glycosylation on plasma half-life and protein activity has also been extensively studied. In the case of tissue plasminogen activator (tPA) the addition of new glycosylation sites decreased the plasma clearance, and improved the potency (Keyt et al., 1994). Glycoengineering has been successfully applied for a number of recombinant proteins and immunoglobulins (Elliott et al., 2003; Raju and Scallon, 2007; Sinclair and Elliott, 2005; Umana et al., 1999). Further, glycosylation influences the stability of immunoglobulins (Mimura et al., 2000; Raju and Scallon, 2006).

In some embodiments, Insl6 proteins or fragments thereof can be fused to the Fc fragment of an IgG (Ashkenazi and Chamow, 1997). The Fc fusion approach has been utilized, for example in the Trap Technology developed by Regeneron (e.g. IL1 trap and VEGF trap). The use of albumin to extend the half-life of peptides has been described in US2004001827A1. Positive effects of albumin have also been reported for Fab fragments and scFv-HSA fusion protein (Smith et al., 2001). It has been demonstrated that the prolonged serum half-life of albumin is due to a recycling process mediated by the FcRn (Anderson et al., 2006; Chaudhury et al., 2003; Smith et al., 2001).

In some embodiments, an Insl6 is conjugated to a biotinylated Fc protein, as disclosed in US application 2010/0209424, which is incorporated herein in its entirety by reference.

As used herein, the term “conjugate” or “conjugation” refers to the attachment of two or more entities to form one entity. For example, the methods of the present invention provide conjugation of an Insl6 polypeptide (i.e. SEQ ID NO: 1 or fragments (e.g., A-domain motifs of amino acids 173-198 of SEQ ID NO:1 and B-domain motif; amino acids 21-53 of SEQ ID NO:1), or derivatives or variants thereof) joined with another entity, for example a moiety such as a first fusion partner that makes the Insl6 protein stable, such as Ig carrier particle, for example IgG1 Fc. The attachment can be by means of linkers, chemical modification, peptide linkers, chemical linkers, covalent or non-covalent bonds, or protein fusion or by any means known to one skilled in the art. The joining can be permanent or reversible. In some embodiments, several linkers can be included in order to take advantage of desired properties of each linker and each protein in the conjugate. Flexible linkers and linkers that increase the solubility of the conjugates are contemplated for use alone or with other linkers as disclosed herein. Peptide linkers can be linked by expressing DNA encoding the linker to one or more proteins in the conjugate. Linkers can be acid cleavable, photocleavable and heat sensitive linkers. Methods for conjugation are well known by persons skilled in the art and are encompassed for use in the present invention.

According to the present invention, an Insl6 polypeptide (i.e. SEQ ID NO: 1 or fragments, derivatives or variants thereof), can be linked to the first fusion partner via any suitable means, as known in the art, see for example U.S. Pat. Nos. 4,625,014, 5,057,301 and 5,514,363, which are incorporated herein in their entirety by reference. For example, an Insl6 polypeptide can be covalently conjugated to the IgG1 Fc, either directly or through one or more linkers. In one embodiment, an Insl6 polypeptide as disclosed herein is conjugated directly to the first fusion partner (e.g. Fc), and in an alternative embodiment, an Insl6 polypeptide as disclosed herein can be conjugated to a first fusion partner (such as IgG1 Fc) via a linker, e.g. a transport enhancing linker.

A large variety of methods for conjugation of an Insl6 polypeptide as disclosed herein with a first fusion partner (e.g. Fc) are known in the art. Such methods are e.g. described by Hermanson (1996, Bioconjugate Techniques, Academic Press), in U.S. Pat. No. 6,180,084 and U.S. Pat. No. 6,264,914 which are incorporated herein in their entirety by reference and include e.g. methods used to link haptens to carriers proteins as routinely used in applied immunology (see Harlow and Lane, 1988, “Antibodies: A laboratory manual”, Cold Spring Harbor Laboratory Press, Cold Spring Harbor, N.Y.). It is recognized that, in some cases, an Insl6 polypeptide can lose efficacy or functionality upon conjugation depending, e.g., on the conjugation procedure or the chemical group utilized therein. However, given the large variety of methods for conjugation the skilled person is able to find a conjugation method that does not or least affects the efficacy or functionality of the entities, such as an Insl6 polypeptide to be conjugated.

Suitable methods for conjugation of an Insl6 polypeptide as disclosed herein with a first fusion partner (e.g. Fc) include e.g. carbodimide conjugation (Bauminger and Wilchek, 1980, Meth. Enzymol. 70: 151-159). Alternatively, a moiety can be coupled to a targeting agent as described by Nagy et al., Proc. Natl. Acad. Sci. USA 93:7269-7273 (1996), and Nagy et al., Proc. Natl. Acad. Sci. USA 95:1794-1799 (1998), each of which are incorporated herein by reference. Another method for conjugating one can use is, for example sodium periodate oxidation followed by reductive alkylation of appropriate reactants and glutaraldehyde crosslinking.

One can use a variety of different linkers to conjugate an Insl6 polypeptide as disclosed herein with a first fusion partner (e.g. Fc), for example but not limited to aminocaproic horse radish peroxidase (HRP) or a heterobiofunctional cross-linker, e.g. carbonyl reactive and sulfhydryl-reactive cross-linker Heterobiofunctional cross linking reagents usually contain two reactive groups that can be coupled to two different function targets on proteins and other macromolecules in a two or three-step process, which can limit the degree of polymerization often associated with using homobiofunctional cross-linkers. Such multi-step protocols can offer a great control of conjugate size and the molar ratio of components.

The term “linker” refers to any means to join two or more entities, for example an Insl6 polypeptide as disclosed herein with a first fusion partner (e.g. Fc). A linker can be a covalent linker or a non-covalent linker Examples of covalent linkers include covalent bonds or a linker moiety covalently attached to one or more of the proteins to be linked. The linker can also be a non-covalent bond, e.g. an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the effector molecule and/or the probe can be modified by oxidation, hydroxylation, substitution, reduction etc. to provide a site for coupling. It will be appreciated that modification which do not significantly decrease the function of an Insl6 polypeptide as disclosed herein or the first fusion partner (e.g. Fc) are preferred.

Targeting.

In some embodiments, an Insl6 protein, or functional fragment, or a homologue for use in the methods and compositions as disclosed herein can be targeted to muscle via a targeting ligand. This is useful for treatment of autoimmune diseases of the muscle, for example, but not limited to myositis. A targeting ligand is a molecule, e.g., small molecule, protein or fragment thereof that specifically binds with high affinity to a target, e.g., a cell-surface marker on a pre-selected cell, such as a surface protein such as a receptor that is present to a greater degree on the pre-selected cell target than on any other body tissue. For example, as described in U.S. Pat. Nos. 5,814,478 and 6,413,740, the MuSK receptor is highly specific to muscle. Accordingly, the cognate ligand agrin, as well as MuSK binding portions thereof is an example of a targeting ligand useful to target the Insl6 agent to muscle. In some embodiments, the targeting ligand is fused an Insl6 protein (e.g., as a second fusion partner), for example fused to an Insl6 polypeptide or functional fragment, homologue, variant thereof. Accordingly, in some embodiments, an Insl6 protein for use in the compositions and methods as disclosed herein can be fused to a Fc and/or optionally also to a targeting molecule. In some embodiments, a nucleic acid encoding a targeting ligand can be fused to a nucleotide encoding an Insl6 protein or fragment or homologue or variant thereof. Another example of a targeting ligand is a group of cadherin domains from a human cadherin. Accordingly, human cadherin domains from, for example, human muscle cadherin may be used in the targeting of agents of the present invention, for example but not limited to targeting an Insl6 polypeptide to target muscle cells. A targeting ligand component attached to an Insl6 polypeptide can include a naturally occurring or recombinant or engineered ligand, or a fragment thereof, capable of binding the pre-selected target cell.

In another embodiment of the invention, a targeting ligand can consists of at least three, four or five muscle cadherin (M-cadherin) domains, or derivatives or fragments thereof, capable of binding specifically to target cells that express homophilic cadherins. (Shimoyama et al. (1998) J. Biol. Chem. 273(16): 1001 1-10018; Shibata et al. (1997) J. Biol. Chem. 272(8):5236-5270). In some embodiments, the targeting ligand comprises at least three cadherin domains from the extracellular domain of human M-cadherin (or biologically active fragments or derivatives thereof that are capable of binding homophilic M-cadherin), fused to an agent of the present invention.

Further examples of targeting ligands also include, but are not limited to, antibodies and portions thereof that specifically bind a pre-selected cell surface protein with high affinity. By “high affinity” is meant an equilibrium dissociation constant of at least molar, as determined by assay methods known in the art, for example, BiaCore analysis. In one embodiment, the targeting ligand may also comprise one or more immunoglobulin binding domains isolated from antibodies generated against a selected tissue-specific surface protein or target tissue-specific receptor. The term “immunoglobulin or antibody” as used herein refers to a mammalian, including human, polypeptide comprising a framework region from an immunoglobulin gene or fragments thereof that specifically binds and recognizes an antigen, which, in the case of the present invention, is a tissue-specific surface protein, a target tissue-specific receptor, or portion thereof. If the intended targeting fusion polypeptide will be used as a mammalian therapeutic, immunoglobulin binding regions should be derived from the corresponding mammalian immunoglobulins. If the targeting fusion polypeptide is intended for non-therapeutic use, such as for diagnostics and ELISAs, the immunoglobulin binding regions may be derived from either human or non-human mammals, such as mice. The human immunoglobulin genes or gene fragments include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (e.g. IgG1, IgG2, etc.). Typically, the antigen-binding region of an antibody will be the most critical in determining specificity and affinity of binding.

An exemplary immunoglobulin (antibody) structural unit of human IgG, comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (VL) and variable heavy chain (VH) refer to these light and heavy chains respectively. Antibodies exist as intact immunoglobulins, or as a number of well-characterized fragments produced by digestion with various peptidases. For example, pepsin digests an antibody below the disulfide linkages in the hinge region to produce F(ab)′2, a dimer of Fab which itself is a light chain joined to VH-CH by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the terms immunoglobulin or antibody, as used herein, also includes antibody fragments either produced by the modification of whole antibodies, or those synthesized de novo using recombinant DNA methodologies (e.g., single chain Fv)(scFv)) or those identified using phase display libraries (see, for example, McCafferty et al. (1990) Nature 348:552-554). In addition, the fusion polypeptides of the invention include the variable regions of the heavy (VH) or the light (VL) chains of immunoglobulins, as well as tissue-specific surface protein and target receptor-binding portions thereof. Methods for producing such variable regions are described in Reiter, et al. (1999) J. Mo!. Biol. 290:685-698.

Methods for preparing antibodies are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. No. 4,946,778; U.S. Pat. No. 4,816,567) can be adapted to produce antibodies used in the fusion polypeptides and methods of the instant invention. Also, transgenic mice, or other organisms such as other mammals, may be used to express human or humanized antibodies. Alternatively phage display technology can be used to identify antibodies, antibody fragments, such as variable domains, and heteromeric Fab fragments that specifically bind to selected antigens.

Screening and selection of preferred immunoglobulins (e.g., antibodies) can be conducted by a variety of methods known to the art: Initial screening for the presence of monoclonal antibodies specific to a tissue-specific or target receptor may be conducted through the use of ELISA-based methods or phage display, for example. A secondary screen is preferably conducted to identify and select a desired monoclonal antibody for use in construction of the tissue-specific fusion polypeptides of the invention. Secondary screening may be conducted with any suitable method known to the art. One method, termed “Biosensor Modification-Assisted Profiling” (“BiaMAP”) (US patent publication 2004/101920), allows rapid identification of hybridoma clones producing monoclonal antibodies with desired characteristics. More specifically, monoclonal antibodies are sorted into distinct epitope-related groups based on evaluation of antibody: antigen interactions.

In some embodiments, an insl6 agent is a protein or polypeptide of Insl6. In some embodiments, an insl6 agent can be a modified RNA (mod-RNA) encoding the Insl6 protein. In some embodiments, the insl6 agent can be an activating RNA agent (RNAa), as defined herein.

In some embodiments, an Insl6 agent as disclosed herein which increases gene expression of the Insl6 gene is a synthetic modified RNAs (herein referred to as “MOD-RNA”), and can be used to induce Insl6 protein expression in tissues. Administration of MOD-RNA results in a very rapid onset of protein expression, with protein expression levels significantly higher, e.g., at least about 2-fold higher, as compared to cells transfected than non-MOD RNA. In some embodiments, the optimal dose range for transfection with MOD-RNA is between 10-30 ng per 1000 cells, and that such a dose is non-toxic to cells.

Synthetic modified RNA's for delivery using the devices and methods as disclosed herein are described in Applications US 2012/0046346, WO 2012/078637, WO2012/019168, WO2012/045075, WO2012/158736, WO2012/045082, US2012/031781, US2012/0251618, each of which are incorporated herein in their entirety by reference. In some embodiments, the synthetic, modified RNA molecule is not expressed in a vector, and the synthetic, modified RNA molecule can be a naked synthetic, modified RNA molecule. In some embodiments, a composition can comprises at least one synthetic, modified RNA molecule present in a lipid complex.

In some embodiments, an insl6 agent which is administered to a subject in the methods and compositions as disclosed herein is a dsRNA which activates gene expression. In particular, in some embodiments, dsRNA which activates gene expression, by a mechanism that has been termed “small RNA-induced gene activation” or “RNAa” can be used (See for example Li, L. C. et al. Proc Natl Acad Sci USA. (2006), 103(46):17337-42 and Li L. C. (2008), “Small RNA-Mediated Gene Activation”, and “RNA and the Regulation of Gene Expression: A Hidden Layer of Complexity”. Caister Academic Press. ISBN 978-1-904455-25-7, which are incorporated herein in their entirety by reference). It has been shown that dsRNAs targeting gene promoters induce potent transcriptional activation of associated genes. Endogenous miRNA that cause RNAa has also been found in humans. Check E. Nature (2007). 448 (7156): 855-858. Another surprising observation is that gene activation by RNAa is long-lasting. Induction of gene expression has been seen to last for over ten days. The prolonged effect of RNAa could be attributed to epigenetic changes at dsRNA target sites.

In some embodiments, an Insl6 agent is a nucleic acid which is an RNA activator (RNAa), wherein oligonucleotide increases the expression of a gene. In some embodiments, increased gene expression of Insl6 by an RNAa agent can be used for treatment of a subject with an autoimmune disease, or immune-related disorder or dysfunction as disclosed herein. Accordingly, in some embodiments, an agent delivered using the device as disclosed herein can be a small activating RNA (RNAa), which is are disclosed in WO06/013559, US2005/0226848A1, WO2009/086428A2, U.S. Pat. No. 6,022,863, which are incorporated herein in their entirety by reference.

Additionally, in some embodiments, an Insl6 agent as disclosed herein is a molecule or compound which increases Insl6 expression by activating the signal pathway which induces the transcription of Insl6, such as activators of Akt signaling, for example, but not limited to SC79, and activator of AKT activity selected from the group consisting of rapamycin, CCl-779, nicotine, Ro-31-8220, carbachol, 4-(methylnitrosamino)-1-(3-pyridyl)-1-butanone (NNK), adrenomedullin (AM) lysophosphatidic acid, platelet activating factor, macrophage simulating factor; sphingosine-1-phosphate, forskolin, chlorophenylthio-cAMP, prostaglandin-E1, and 8-bromo-cAMP, insulin, insulin growth factor-1, platelet derived growth factor and granulocyte colony-stimulating factor (G-CSF).

### Methods to Treat Autoimmune Diseases and Immune-Related Diseases and Disorders

One aspect of the present invention provides methods for treating autoimmune diseases and immune-related disorders in a subject. In some embodiments, the present invention provides methods and compositions to treat a subject with a muscle autoimmune disease, such as myositis, e.g., polymyositis (PM), dermatomysitis (DM), and inclusion body myositis (IBM), or a subject at risk of developing or having myositis. In some embodiments, a subject which exhibits at least one symptom of an autoimmune disease (e.g., a muscle autoimmune disease, e.g., myositis) is administered an Insl6 protein or agent according to the methods as disclosed herein. In such an embodiment, the method comprises administration of an effective amount of a pharmaceutical composition comprising a gene and/or gene product (i.e. proteins) of Insl6 or a functional fragment or functional derivative or agonists thereof to a subject in need thereof. In some embodiments, the pharmaceutical compositions comprise an agonist of Insl6.

In some embodiments, the present invention provides a method to treat a subject with, or at risk of an autoimmune disease by administering a pharmaceutical composition comprising an Insl6 protein (SEQ ID: NO: 1) or a functional fragment, or a homologue or a variant thereof to the subject. In an alternative embodiment, the present invention provides a method to treat a subject with, or at risk of an autoimmune disease by administering a pharmaceutical composition comprising a nucleic acid corresponding to SEQ ID: NO: 2 or a fragment or variant thereof which encodes the Insl6 protein (SEQ ID NO: 1) or a fragment thereof to the subject. In an alternative embodiment, the present invention provides a method to treat a subject with, or at risk of an autoimmune disease by administering a pharmaceutical composition comprising a nucleic acid which is a MOD-RNA which encodes a Insl6 protein (SEQ ID: NO: 1) or a homologue or fragment thereof to the subject. In some embodiments, a MOD-RNA nucleic acid encodes the A-domain of Insl6 (amino acids 173-198 of SEQ ID NO:1) and/or the B-domain of Insl6 (amino acids 21-53 of SEQ ID NO:1), or functional fragments thereof.

A pharmaceutical composition comprising an Insl6 protein, or a functional fragment or derivative thereof will result in reduction of immune cells in the subject, e.g., a reduction of immune cells which express at least one or more of: CD11b, CD4, CD8, and/or a reduction in pro-inflammatory cytokines selected from, but not limited to, TNFα or MCP-1. In some embodiments, composition comprising an Insl6 protein results in a decrease in immune cells (e.g., T-cells expressing CD4 and/or CD8, or macrophages expressing CD11b) in the subject by at least 1%, or by at least about 5%, or by at least about 10%, or by at least 20%, or by at least 50%, in the presence of Insl6 protein as compared to the absence of Insl6.

In some embodiments, composition comprising an Insl6 agent, (e.g., Insl6 protein or nucleic acid encoding an Insl6 protein) results in a decrease in pro-inflammatory cytokines (e.g., but not limited to, TNFα, MCP-1 and other pro-inflammatory cytokines) in the subject by at least 1%, or by at least about 5%, or by at least about 10%, or by at least 20%, or by at least 50%, in the presence of Insl6 protein as compared to the absence of Insl6. In some embodiments, a pro-inflammatory cytokine is selected from any one or a combination of: cytokines, lymphokines, monokines, stem cell growth factors, lymphotoxins, hematopoietic factors, colony stimulating factors (CSF), interferons (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, transforming growth factor (TGF), TGF-α, TGF-β, insulin-like growth factor (IGF), erythropoietin, thrombopoietin, tumor necrosis factor (TNF), TNF-α, TNF-β, mullerian-inhibiting substance (MIS), mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-.alpha., interferon-.beta., interferon-.gamma., S1 factor, IL-1, IL-1 cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21, IL-23, IL-25, LIF, kit-ligand, FLT-3, angiostatin, thrombospondin and endostatin. Accordingly, in some embodiments, the Insl6 agents as disclosed herein can be used in compositions and methods to treat sepsis.

In some embodiments, a pro-inflammatory cytokine can be selected from any or a combination of interleukin-1β (IL-1β), tumor necrosis factor-α (TNF-α), interleukin-6 (IL-6), interleukin-8 (IL-8), interferon-γ (IFN-γ), vascular endothelial growth factor (VEGF), leukemia inhibitory factor (LIF), monocyte chemoattractant protein-1 (MCP-1), RANTES, interleukin-10 (IL-10), interleukin-12 (IL-12), matrix metalloproteinase 2 (MMP2), IP-10, macrophage inflammatory protein 1α (MIP1α) and/or macrophage inflammatory protein 1β (MIP1β).

In particular, another aspect of the present invention provides methods for treating an inflammatory disease or disorder in a subject. In some embodiments, the present invention provides methods and compositions to treat a subject with an inflammatory disease or disorder, or a subject at risk of developing or having an inflammatory disease or disorder, e.g., but not limited to, sepsis. In such an embodiment, the method comprises administration of an effective amount of a pharmaceutical composition comprising an insl6 agent, e.g., a gene and/or gene product (i.e. protein) of Insl6 or a functional fragment or functional derivative or agonists thereof, or an Insl6-fusion protein as disclosed herein to a subject in need thereof. In some embodiments, the pharmaceutical compositions comprise an agonist of Insl6.

In some embodiments, the inflammatory disorder is sepsis. In another embodiment, an inflammatory condition or disease is ileus, and/or endotoxic shock and/or sepsis. In some embodiments, the inflammatory disease or disorder is selected from any one or a combination of inflammatory disorder is selected from the group consisting of, but not limited to, appendicitis, peptic ulcer, gastric ulcer, duodenal ulcer, peritonitis, pancreatitis, ulcerative colitis, pseudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumotransmicroscopicsilicovolcanoconiosis, alvealitis, bronchiolitis, pharyngitis, pleurisy, sinusitis, influenza, respiratory syncytial virus infection, HIV infection, hepatitis B virus (HBV) infection, hepatitis C virus (HCV) infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease (GVHD), Type I diabetes, ankylosing spondylitis, Berger's disease, Reiter's syndrome and Hodgkin's disease.

In some embodiments, the reduction of immune cells, and/or reduction in pro-inflammatory cytokines induced by an Insl6 agent can be compared with the amount of immune cells, and/or amount of pro-inflammatory cytokines with a positive control. In some embodiments, the positive control is an inhibitor of Insl6, for example a neutralizing antibody of Insl6 or inhibitory nucleic acid of Insl6, for example RNAi of Insl6 or a dominant negative form of Insl6.

Another aspect of the present invention relates to use of an insl6 agent, e.g., an insl6 protein or functional fragment thereof to modify (e.g., decrease or reduce) the activity of Regulatory T cells. Without wishing to be bound by theory, regulatory T cells (T_{reg}), are also referred to as suppressor T cells, are a specialized subpopulation of T cells which suppresses activation of the immune system and thereby maintains tolerance to self-antigens. Regulatory T cells have a critical role in the vertebrate immune system, and function to have an immunosuppressive effect, which can be used to treat autoimmune diseases and manipulated to facilitate organ transplantation and cancer immunotherapy.

Regulatory T cells come in many forms, including those that express CD4, CD25, and Foxp3 (CD4+CD25+ regulatory T cells, or “T_{regs}”). T_{regs} cells are involved in shutting down immune responses after they have successfully tackled invading organisms, and also in regulating immune responses that may potentially attack one's own tissues (autoimmunity).

CD4+Foxp3+ regulatory T cells have been referred to as “naturally-occurring” regulatory T cells to distinguish them from “suppressor” T cell populations that are generated in vitro. The regulatory T cell also includes suppressive T cell populations, including Tr1, Th3, CD8+CD28−, and Qa-1 restricted T cells.

T-regulatory (Treg) cells are a subpopulation of CD4+ T cells, and express CD25, which is the alpha chain of the receptor for interleukin-2 (IL-2); and also express Foxp3 transcription factor that alters the expression of many genes—enhancing some; suppressing others; and also expresses CTLA-4 (“cytotoxic T-lymphocyte-associated antigen 4”), a cell-surface protein designated.

Like other T cells, Treg cells also express the alpha-beta T-cell receptor for antigen (αβ TCR) and can only be activated if it binds to the peptide-class II MHC molecule for which it is specific and the cell also receives co-stimulation from B7 molecules (also known as CD80 and CD86) on the antigen-presenting cell. However, if activated, Treg cells begin to secrete large amounts of interleukin 10 (IL-10) and TGF-β. Treg cells are powerful immunosuppressants and may be one of the mechanisms by which Treg cells inhibit Th1 helper cells for cell-mediated immunity (including graft-versus-host disease) and inflammation; Th2 helper cells involved in antibody production; Th17 cells; natural killer (NK) cells; and the action of CD8+ cytotoxic T lymphocytes (CTL). Treg cells may also kill the various kinds of effector T cells by binding to them and secreting granzymes and perforins.

But perhaps the most important suppressor mechanism is the effect of Treg cells on antigen-presenting cells (APCs) like dendritic cells. The CTLA-4 molecules on Treg cells bind tightly to the B7 molecules on the APCs, strip them off the APC, engulf them by endocytosis and destroy them. Having lost their B7 (CD80 and 86) molecules, the APCs can no longer present “signal 2” to activate effector T cells.

Treg cells respond to the presence of interleukin-2 (IL-2) by rapid proliferation. Because IL-2 is secreted by effector T cells, this provides a negative-feedback mechanism: inflammatory T-cell activity (e.g., by Th1 cells) is restrained by the resulting expansion of Treg cells.

### Autoimmune Diseases:

In some embodiments, the compositions and methods as disclosed herein can be used to treat an autoimmune disease, e.g., a muscle autoimmune disease such as myositis, or other muscle autoimmune diseases such as myocarditis and the like. In some embodiments, the compositions and methods as disclosed herein can be used to treat an autoimmune disease such as lupus, multiple sclerosis, diabetes and the like. In some embodiments, the compositions and methods as disclosed herein can be used to treat an autoimmune disease where the subject is determined to have abnormal T-regulatory cell levels (e.g., high levels) as compared to a normal subject, e.g., a healthy subject without an autoimmune disease. In some embodiments, the compositions and methods as disclosed herein can be used to treat an autoimmune disease where the subject is determined to have high or elevated levels of auto-immune antibodies as compared to a normal subject, e.g., a healthy subject without an autoimmune disease.

In some embodiments, the compositions and methods as disclosed herein can be used to treat an autoimmune disease selected from any one or a combination of the autoimmune-related disease or disorder such as rheumatoid arthritis, multiple sclerosis (MS), systemic lupus erythematosus (SLE), autoimmune myocarditis, sepsis, Graves' disease (overactive thyroid), Hashimoto's thyroiditis (underactive thyroid), Type 1 diabetes mellitus, celiac disease, Crohn's disease and ulcerative colitis, Guillain-Barre syndrome, primary biliary sclerosis/cirrhosis, sclerosing cholangitis, autoimmune hepatitis, Raynaud's phenomenon, scleroderma, Sjogren's syndrome, Goodpasture's syndrome, Wegener's granulomatosis, polymyalgia rheumatica, temporal arteritis/giant cell arteritis, chronic fatigue syndrome CFS), psoriasis, autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, Wegener's granulomatosis and fibromyalgia (FM).

In one embodiment, the immune-related disease or disorder is organ transplantation rejection or graft-vs-host-disease (GVH). The method encompassed preventing as well as treating organ transplantation rejection in a subject who will be receiving or is an recipient of a donor organ. The method disclosed herein comprises administering to the mammal a therapeutically-effective amount of an Insl6 protein or a fragment or fusion protein thereof as disclosed herein. Transplant rejection occurs when the immune system of the recipient of a transplant attacks the transplanted donor organ or tissue such as the heart, lungs, pancreas, liver, and kidneys. This is because a normal healthy human immune system can distinguish foreign tissues and attempts to destroy them, just as it attempts to destroy infective organisms such as bacteria and viruses. Acute organ rejection is generally mediated by T cell responses to proteins from the donor organ which differ from those found in the recipient. The development of T cell responses first occurs several days after a transplant if the patient is not taking immunosuppressant drugs. Acute organ rejection is caused by mismatched human leukocyte antigens (HLA) antigens that are present on all cells. HLA antigens are polymorphic therefore the chance of a perfect match is extremely rare. The reason that acute rejection occurs a week after transplantation is because the T-cells involved in rejection must be activated first by the foreign HLA, then differentiate and the antibodies in response to the allograft must be produced before rejection is initiated. These activated T-cells cause the graft cells to lyse or they produce cytokines that recruit other inflammatory cells, eventually causing necrosis of donor tissue. Endothelial cells in vascularized grafts such as kidneys are some of the earliest victims of acute rejection. Damage to the endothelial lining is an early predictor of irreversible acute graft failure. The new organ is then incapable of working at full efficiency, and symptoms of rejection become apparent to the transplant recipient. These symptoms of rejection are very similar to the symptoms of organ failure. Physicians skilled in the art can recognize and diagnose transplantation rejection. A biopsy of the transplanted organ can confirm that it is being rejected.

In some aspects, the autoimmune-related disease or disorder that the method described herein can be but is not limited to an organ-specific autoimmune diseases, such as inflammatory arthritis, type 1 diabetes mellitus, psoriasis, inflammatory bowel diseases, and vasculitis, allergic inflammation, such as allergic asthma, atopic dermatitis, and contact hypersensitivity.

In some embodiments, an autoimmune disease is selected from the group consisting of rheumatoid arthritis, spondylarthropathies, Sjogren's syndrome, polymyositis (PS), scleroderma, dermatomyositis (DM), autoimmune polyneuritis, myasthenia gravis, insulin-resistant diabetes, autoimmune adrenal insufficiency (Addison's disease), autoimmune oophoritis, autoimmune orchitis, autoimmune hemolytic anemia, paroxysmal cold hemoglobinuria, autoimmune thrombocytopenia, pernicious anemia, pure red cell anemia, autoimmune coagulopathies, pemphigus and other bullous diseases, psoriasis, rheumatic fever, vasculitis, postcardiotomy syndrome (Dressler's syndrome), billiary cirrhosis, and autoimmune hepatitis.

In some embodiments, an autoimmune disease is selected from any or a combination of the group of autoimmune diseases consisting of: acute immune thrombocytopenia, chronic immune thrombocytopenia, Sydenham's chorea, myasthenia gravis, lupus nephritis, rheumatic fever, polyglandular syndromes, bullous pemphigoid, pemphigus vulgaris, Henoch-Schonlein purpura, post-streptococcal nephritis, erythema nodosum, Takayasu's arteritis, sarcoidosis, erythema multiforme, IgA nephropathy, polyarteritis nodosa, ankylosing spondylitis, thromboangitis obliterans, primary biliary cirrhosis, thyrotoxicosis, chronic active hepatitis, polychondritis, pemphigus vulgaris, membranous nephropathy, amyotrophic lateral sclerosis, tabes dorsalis, giant cell arteritis/polymyalgia, pernicious anemia, rapidly progressive glomerulonephritis, and fibrosing alveolitis.

Insl6 proteins, and/or Insl6 agents (e.g., agents which increase the expression of Insl6 can be used to treat immune dysregulation disease and related autoimmune diseases. Immune diseases may include acute immune thrombocytopenia, Addison's disease, adult respiratory distress syndrome (ARDS), agranulocytosis, allergic conditions, allergic encephalomyelitis, allergic neuritis, amyotrophic lateral sclerosis (ALS), ankylosing spondylitis, antigen-antibody complex mediated diseases, anti-glomerular basement membrane disease, anti-phospholipid antibody syndrome, aplastic anemia, arthritis, asthma, atherosclerosis, autoimmune disease of the testis and ovary, autoimmune endocrine diseases, autoimmune myocarditis, autoimmune neutropenia, autoimmune polyendocrinopathies, autoimmune polyglandular syndromes (or polyglandular endocrinopathy syndromes), autoimmune thrombocytopenia, Bechet disease, Berger's disease (IgA nephropathy), bronchiolitis obliterans (non-transplant), bullous pemphigoid, pemphigus vulgaris, Castleman's syndrome, Celiac sprue (gluten enteropathy), central nervous system (CNS) inflammatory disorders, chronic active hepatitis, chronic immune thrombocytopenia dermatomyositis, colitis, conditions involving infiltration of T cells and chronic inflammatory responses, coronary artery disease, Crohn's disease, cryoglobulinemia, dermatitis, dermatomyositis, diabetes mellitus, diseases involving leukocyte diapedesis, eczema, encephalitis, erythema multiforme, erythema nodosum, Factor VIII deficiency, fibrosing alveolitis, giant cell arteritis, glomerulonephritis, Goodpasture's syndrome, graft versus host disease (GVHD), granulomatosis, Grave's disease, Guillain-Barre Syndrome, Hashimoto's thyroiditis, hemophilia A, Henoch-Schonlein purpura, idiopathic hypothyroidism, immune thrombocytopenia (ITP), IgA nephropathy, IgA nephropathy, IgM mediated neuropathy, immune complex nephritis, immune hemolytic anemia including autoimmune hemolytic anemia (ARIA), immune responses associated with acute and delayed hypersensitivity mediated by cytokines and T-lymphocytes, immune-mediated thrombocytopenias, juvenile onset diabetes, juvenile rheumatoid arthritis, Lambert-Eaton Myasthenic Syndrome, large vessel vasculitis, leukocyte adhesion deficiency, leukopenia, lupus nephritis, lymphoid interstitial pneumonitis (HIV), medium vessel vasculitis, membranous nephropathy, meningitis, multiple organ injury syndrome, multiple sclerosis, myasthenia gravis, osteoarthritis, pancytopenia, pemphigoid bullous, pemphigus vulgaris, pernicious anemia, polyarteritis nodosa, polychondritis, polyglandular syndromes, polymyalgia, polymyositis, post-streptococcal nephritis, primary biliary cirrhosis, primary hypothyroidism, psoriasis, psoriatic arthritis, pure red cell aplasia (PRCA), rapidly progressive glomerulonephritis, Reiter's disease, respiratory distress syndrome, responses associated with inflammatory bowel disease, Reynaud's syndrome, rheumatic fever, rheumatoid arthritis, sarcoidosis, scleroderma, Sjogren's syndrome, solid organ transplant rejection, Stevens-Johnson syndrome, stiff-man syndrome, subacute thyroiditis, Sydenham's chorea, systemic lupus erythematosus (SLE), systemic scleroderma and sclerosis, tabes dorsalis, Takayasu's arteritis, thromboangitis obliterans, thrombotic thrombocytopenic purpura (TTP), thyrotoxicosis, Hashimoto's thryoidosis, toxic epidermal necrolysis, tuberculosis, Type I diabetes, ulcerative colitis, uveitis, vasculitis (including ANCA) and Wegener's granulomatosis.

In particular embodiments, an autoimmune disease which can be treated with an Insl6 agent by the methods as disclosed herein include Addison's disease, Celiac disease—sprue (gluten-sensitive enteropathy), Dermatomyositis, Graves disease, Hashimoto's thyroiditis, Multiple sclerosis, Myasthenia gravis, Pernicious anemia; Reactive arthritis, Rheumatoid arthritis, Sjogren syndrome, Systemic lupus, erythematosus, and Type I diabetes.

In certain embodiments, an autoimmune disease or autoimmune disorder is a vascular disorder, the vascular disorder may include any vascular disease or disorder which comprises an autoimmune element, for example one which is caused by an autoimmune response. Exemplary vascular disorders include one or more of Raynaud's disease and phenomenon, anterior uveitis, vasculitis, obliterative vascular disorder, atheroma formation (i.e., arteriosclerosis), arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, atherosclerotic heart disease, reperfusion injury, cardiac conduction disturbances, myocarditis, and myocardial infarction. In certain embodiments, the vascular disorder is selected from one or more of Raynaud's disease and phenomenon, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, and cardiac conduction disturbances. In certain embodiments, the vascular disorder is selected from one or more of Raynaud's disease and phenomenon, anterior uveitis, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, atherosclerotic heart disease, cardiac conduction disturbances, and myocardial infarction. In certain embodiments, the vascular disorder is selected from one or more of Raynaud's disease and phenomenon, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, atherosclerotic heart disease, cardiac conduction disturbances, and myocardial infarction. In certain embodiments, the vascular disorder is selected from one or more of Raynaud's disease and phenomenon, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, and cardiac conduction disturbances.

In some embodiments, a subject with an autoimmune disease has at least one symptom selected from the following: Fatigue, Fever, General ill-feeling (malaise). In some embodiments, a subject is diagnosed or identified to have an autoimmune disease by at least one of the following tests: Antinuclear antibody tests, Autoantibody tests, CBC, C-reactive protein (CRP), Erythrocyte sedimentation rate (ESR).

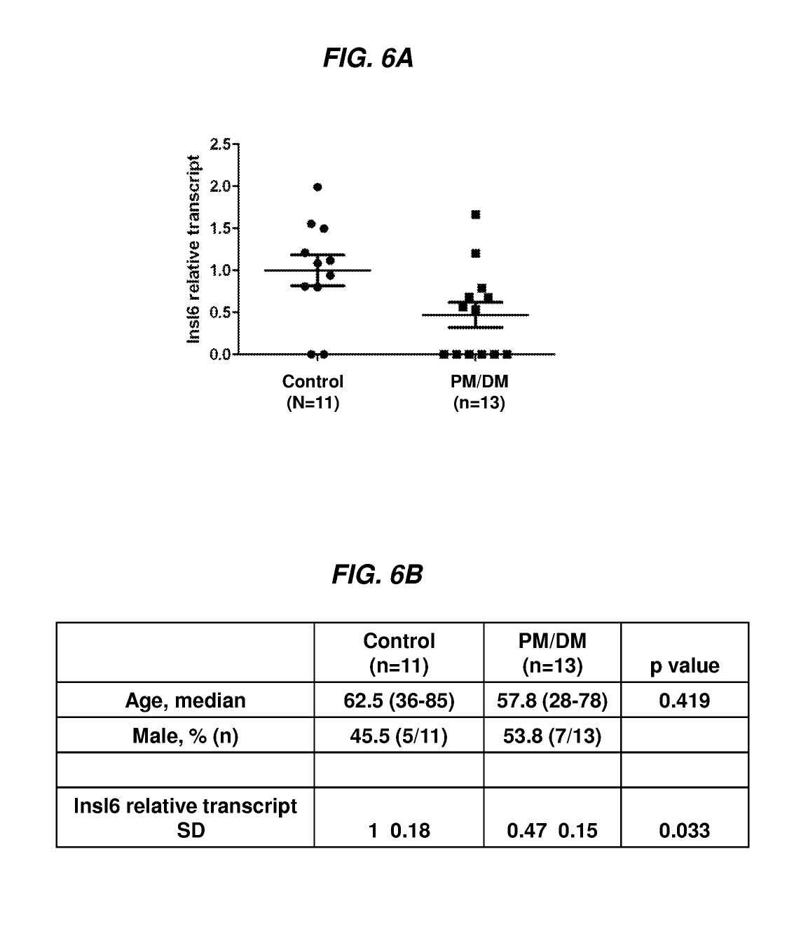

The methods of the present invention are also useful to treat any condition which is results from a deficiency in Insl6 protein or function in a subject, for example any condition due to a deficiency in Insl6 or which may be improved by increased levels of Insl6 protein, the method comprising administering an effective amount of pharmaceutical composition comprising an Insl6 agent, e.g., a Insl6 protein or functional fragment or variant thereof, where the effective amount is sufficient to alleviate at least one or some of the symptoms associated with the lack of, or reduced expression level of Insl6. Such diseases include, for example, but are not limited to autoimmune diseases such as myositis as disclosed herein in FIG. 6A and the Examples.

In alternative embodiments, the present invention relates to a method of treating a subject with high levels of Insl6, or increased levels of T-regulatory (T_{reg}) cells as compared to a normal healthy subject, with an antagonist or inhibitor of Insl6, such as a RNAi to Insl6, or an antibody which specifically binds and neutralizes Insl6. RNAi agents and anti-Insl6 antibodies are well known in the art and are encompassed for use in the methods of the present invention.

Subjects Amenable to Treatment with the Insl6 Agents

In some embodiments, a subject amenable to treatment with the methods and compositions as disclosed herein has an autoimmune disease, or has at least one symptom of a known autoimmune disease as disclosed herein. As disclosed herein, the inventors have discovered that subjects with myositis have lower levels of Insl6 expression in the muscle than normal healthy controls (see FIG. 6).

Another aspect of the present invention relates to the diagnosis of a subject with myositis, where the level of Insl6 protein and/or gene expression is measured in a biological sample obtained from the subject, and if the level of Insl6 protein and/or gene expression lower, e.g., decreased by a statistically significant amount relative to an reference level of Insl6 expression, then the subject is likely to have, or is at risk of developing myositis. In some embodiments, the reference level is the level in a comparable (e.g., age matched, tissue matched, gender matched) biological sample from a normal or healthy subject whom does not have myositis or an autoimmune disease. In some embodiments, the biological sample is a muscle biopsy.

In some embodiments, the present invention relates to a method of identifying an individual suitable for treatment with an Insl6 agent as disclosed herein, the method comprising a step of detecting in a biological sample taken from the subject presenting a symptom of an immune disease or disorder for the presence of level of the Insl6 protein of SEQ ID NO: 1 or gene transcript of SEQ ID NO: 2, where a lower level of Insl6 protein or gene transcript as compared to a reference level of Insl6 protein or gene transcript in the subject indicates a risk of the subject having or at risk of an autoimmune disease, e.g., myositis, and in some embodiments, the subject should be administered a composition comprising an Insl6 agent according to the methods as disclosed herein. In some embodiments, the method of detecting the level of the Insl6 protein and/or Insl6 gene transcript (e.g, mRNA) is by quantitative methods, such as by using antibodies which specifically bind to the Insl6 protein, e.g., by using immunoassays, such as ELISA for detecting Insl6 protein levels, or RT-PCT, e.g., quantitative RT-PCR for detecting levels of Insl6 mRNA.

Accordingly, in one aspect, the present invention relates to a method for determining prognosis and/or diagnosis of a subject with myositis. In one embodiment, the method comprises determining the level of Insl6 in a biological sample from the subject, and comparing the determined level of Insl6 with a reference level of Insl6, wherein a decrease in the determined level of at least about 0.2-fold as compared to the prior level, indicates the subject has myositis or is at risk of developing myositis.

As used herein the term “reference level” refers to a Insl6 expression level in a particular biological sample which provides a baseline against which to measure a Insl6 expression level from the test biological sample. As an illustrative example, the reference level for Insl6 expression can be calculated as the average level of the Insl6 gene and/or protein expression from a plurality of biological samples obtained from a plurality of subjects with similar demographics (i.e. age, gender, weight, ethnicity and the like). As another illustrative example only, a reference level for Insl6 expression can be from a plurality of subjects that do not have risk of, or have an autoimmune disease, such as myositis and/or an immune-related disorder. As another illustrative example only, a reference level for Insl6 expression can be from the same subject taken at an earlier timepoint. Typically, a reference level is normalized to “0” value, and a decrease, for example at least about a 30% decrease in the Insl6 expression level measured as disclosed herein relative to the reference level would indicate a subject would have, or at risk of developing an autoimmune disease, e.g., myositis. A reference Insl6 expression level can be from an individual not affected by a given pathology (i.e., not affected with an autoimmune disease, immune-related disorder or myositis), or, alternatively, from the same individual being tested, where the biological sample for the reference Insl6 expression was taken at an at least one earlier time point (i.e. t₀, t₁, t₂ etc). A reference can also be a pooled sample, taken from a plurality of individuals not affected by the pathology in question (i.e., not affected with an autoimmune disease, immune-related disorder or myositis). Where appropriate, a reference can also be a fixed reference level of a Insl6 expression level, where a test Insl6 expression level below the fixed reference level (i.e. at least about 30% below the fixed reference level) a subject is identified as having, or at risk of developing an autoimmune disease, e.g., myositis (i.e. a positive myositis test result). It is preferred that a reference sample be from an individual or group of individuals of similar characteristics to the tested individual, e.g., that the reference be taken from individuals of similar age, gender, rave or ethnic background, etc. In some embodiments, other reference levels can also be used, for example a positive reference Insl6 expression level can be used as a positive control for a subject having a risk of developing or having an autoimmune disease, e.g., myositis. Typically, where a positive reference level is used, if the insl6 expression level in the test biological sample as disclosed herein is substantially the same or close in the value of the positive reference Insl6 expression level, it would indicate a positive test result for a subject having, or at risk of developing an autoimmune disease, e.g., myositis.

In another respect, the present invention relates to a method for determining the relative level of insl6 in a subject or an individual, as it relates to myositis in the individual. The method comprises determining the amount of Insl6 in a biological sample from the individual and comparing the determined amount of Insl6 to an appropriate control, e.g., a reference level from a normal healthy subject.

In some embodiments, where the level of Insl6 expression in the biological sample is at least 30% below a reference Insl6 expression level, is indicative of a subject which has, or is likely to develop myositis. In some embodiments where the level of Insl6 expression in the biological sample is at least 35%, or at least 40% or at least 45% or above 45% below or lower the reference Insl6 expression level, it is indicative of a subject which has, or is likely to develop myositis.

In another embodiment, the amount of insl6 is determined by quantitative detection of Insl6 protein/polypeptide. In one embodiment, the detection of Insl6 protein or polypeptide is by immunoassay, e.g., using an antibody which specifically binds to the Insl6 protein. In one embodiment, the immuno assay is Western blot analysis or ELISA. The biological sample may be blood, serum, plasma, sputum, saliva, stool, tissue, urine, stool, lymph fluid, tears, or milk. The tissue may be from an organ, skeletal muscle, or neuronal tissue. The tissue may be cardiac tissue. The tissue may be obtained from a biopsy, e.g., a muscle biopsy. Insl6 levels can also be determined through quantitative detection of Insl6 mRNA, e.g., by RT-PCR and the like.

In some embodiments, the Insl6 polypeptide expression level is measured by immuno assay, for example western blot analysis or ELISA, or a highthrough-put protein detection method, for example but are not limited to automated immunohistochemistry apparatus, for example, robotically automated immunohistochemistry apparatus which in an automated system section the tissue or biological sample specimen, prepare slides, perform immunohistochemistry procedure and detect intensity of immunostaining, such as intensity of anti-Insl6 antibody staining in the biological sample or tissue and produce output data. Examples of such automated immunohistochemistry apparatus are commercially available, for example such Autostainers 360, 480, 720 and Labvision PT module machines from LabVision Corporation, which are disclosed in U.S. Pat. Nos. 7,435,383; 6,998,270; 6,746,851, 6,735,531; 6,349,264; and 5,839; 091 which are incorporated herein in their entirety by reference. Other commercially available automated immunohistochemistry instruments are also encompassed for use in the present invention, for example, but not are limited BOND™ Automated Immunohistochemistry & In Situ Hybridization System, Automate slide loader from GTI vision. Automated analysis of immunohistochemistry can be performed by commercially available systems such as, for example, IHC Scorer and Path EX, which can be combined with the Applied spectral Images (ASI) CytoLab view, also available from GTI vision or Applied Spectral Imaging (ASI) which can all be integrated into data sharing systems such as, for example, Laboratory Information System (LIS), which incorporates Picture Archive Communication System (PACS), also available from Applied Spectral Imaging (ASI) (see world-wide-web: spectral-imaging.com). Other a determination module can be an automated immunohistochemistry systems such as NexES® automated immunohistochemistry (IHC) slide staining system or BenchMark® LT automated IHC instrument from Ventana Discovery SA, which can be combined with VIAS™ image analysis system also available Ventana Discovery. BioGenex Super Sensitive MultiLink® Detection Systems, in either manual or automated protocols can also be used as the detection module, preferably using the BioGenex Automated Staining Systems. Such systems can be combined with a BioGenex automated staining systems, the i6000™ (and its predecessor, the OptiMax® Plus), which is geared for the Clinical Diagnostics lab, and the GenoMx 6000™, for Drug Discovery labs. Both systems BioGenex systems perform “All-in-One, All-at-Once” functions for cell and tissue testing, such as Immunohistochemistry (IHC) and In Situ Hybridization (ISH).

In some embodiments, the level of Insl6 polypeptide can be determined using any known systems for automated protein expression analysis, including for example, but not limited Mass Spectrometry systems including MALDI-TOF, or Matrix Assisted Laser Desorption Ionization-Time of Flight systems; SELDI-TOF-MS ProteinChip array profiling systems, e.g. Machines with Ciphergen Protein Biology System II™ software; systems for analyzing gene expression data (see for example U.S. 2003/0194711); systems for array based expression analysis, for example HT array systems and cartridge array systems available from Affymetrix (Santa Clara, Calif. 95051) AutoLoader, Complete GeneChip® Instrument System, Fluidics Station 450, Hybridization Oven 645, QC Toolbox Software Kit, Scanner 3000 7G, Scanner 3000 7G plus Targeted Genotyping System, Scanner 3000 7G Whole-Genome Association System, GeneTitan™ Instrument, GeneChip® Array Station, HT Array; an automated ELISA system (e.g. DSX® or DS2® form Dynax, Chantilly, Va. or the ENEASYSTEM III®, Triturus®, The Mago® Plus); Densitometers (e.g. X-Rite-508-Spectro Densitometer®, The HYRYS™ 2 densitometer); automated Fluorescence insitu hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, Becton Dickinson); radio isotope analyzers (e.g. scintillation counters).

In some embodiments, the Insl6 expression level is the Insl6 gene expression level, for example the expression level of Insl6 mRNA corresponding to the nucleotide of SEQ ID NO: 2.

### Administration of Pharmaceutical Compositions

An Insl6 agent, e.g., insl6 protein, Fc-fusion protein or derivative or functional fragment thereof can be administered by any route known in the art or described herein, for example, oral, parenteral (e.g., intravenously or intramuscularly), intraperitoneal, rectal, cutaneous, nasal, vaginal, inhalant, skin (patch), or ocular. The Insl6 agent, e.g., insl6 protein, Fc-fusion protein or derivative or functional fragment protein may be administered in any dose or dosing regimen.

With respect to the therapeutic methods of the invention, it is not intended that the administration of the Insl6 protein or polynucleotide encoding such an Insl6 protein or fusion protein or functional fragment thereof be limited to a particular mode of administration, dosage, or frequency of dosing; the present invention contemplates all modes of administration, including intramuscular, intravenous, intraperitoneal, intravesicular, intraarticular, intralesional, subcutaneous, or any other route sufficient to provide a dose adequate to treat an autoimmune disease or immune-related disorder as disclosed herein. An effective amount, e.g., a therapeutically effective dose of an insl6 agent may be administered to the patient in a single dose or in multiple doses. When multiple doses are administered, the doses may be separated from one another by, for example, one hour, three hours, six hours, eight hours, one day, two days, one week, two weeks, or one month. For example, a composition comprising an insl6 agent can be administered for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity.

While the attending physician ultimately will decide the appropriate amount and dosage regimen, an effective amounts of an Insl6 agent, e.g., insl6 protein, insl6-Fc-fusion protein or derivative or functional fragment thereof can provided at a dose of 0.0001, 0.01, 0.01 0.1, 1, 5, 10, 25, 50, 100, 500, or 1,000 mg/kg. Effective doses may be extrapolated from dose-response curves derived from in vitro or animal model test bioassays or systems.

Dosages for a particular patient or subject can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the Insl6 agent, e.g., insl6 protein, Insl6-Fc-fusion protein or functional derivatives or functional fragments thereof as disclosed herein, and the condition of the patient, the autoimmune disease to be treated, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising an Insl6 agent, e.g., insl6 protein, Insl6-Fc-fusion protein or functional derivatives or functional fragments thereof are optionally tested in one or more appropriate in vitro and/or in vivo animal models of disease, such a CIM mouse model as disclosed herein, or other models of autoimmune disease commonly known to persons of ordinary skill in the art, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of an Insl6 agent, e.g., insl6 protein, Insl6-Fc-fusion protein or functional derivatives or functional fragments thereof at various concentrations, e.g., as applied to the mass and overall health of the patient. Administration can be accomplished via single or divided doses.

In determining the effective amount of Insl6 agent, e.g., insl6 protein, Insl6-Fc-fusion protein or functional derivatives or functional fragments thereof to be administered in the treatment or prophylaxis of a disease, the physician evaluates circulating plasma levels, formulation toxicities, and progression of the disease. The selected dosage level will also depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

In some embodiments, Insl6 agents as disclosed herein can be administered at a dose in accordance with good medical practice, taking into account the clinical condition of the individual patient, the site and method of administration, scheduling of administration, patient age, sex, body weight and other factors known to medical practitioners.

Dosage regimens of a composition comprising an Insl6 agent, e.g., Insl6 protein or functional fragment or variant thereof as disclosed herein can be adjusted to provide the optimum desired response (e.g. a therapeutic or prophylactic response). For example, a single bolus can be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

Furthermore, actual dosage levels of Insl6 agents in a pharmaceutical composition can be varied so as to obtain an amount of the active ingredient which is effective to achieve the desired therapeutic response for a particular subject, composition, and mode of administration, without being toxic to the subject. A pharmaceutical composition comprising an Insl6 agent, e.g., Insl6 protein or functional fragment or variant thereof as disclosed herein can be a “therapeutically effective amount” and/or a “prophylactically effective amount”. In general, a suitable daily dose of a composition comprising an Insl6 agent, e.g., Insl6 protein or functional fragment or variant thereof as disclosed herein will be that amount of the Insl6 agent which is the lowest dose effective to produce a therapeutic effect, such as a reduction of a symptom of an autoimmune disease, for example, a reduction of immune cells which express CD4 and/or CD8 as disclosed herein, or a reduction of at least one pro-inflammatory cytokine in the subject. Such an effective dose will generally depend upon the factors described above.

If desired, the effective daily dose of a composition comprising an Insl6 agent, e.g., Insl6 protein or functional fragment or variant thereof can be administered as two, three, four, five, six or more sub-doses administered separately at appropriate intervals throughout the day, optionally, in unit dosage forms.

It is to be noted that dosage values may vary with the type and severity of the autoimmune disease or immune-related disorder or dysfunction to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The efficacy and toxicity of the compound can be determined by standard pharmaceutical procedures in cell cultures or experimental animals, e.g., ED50 (the dose is effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. An appropriate experimental model which can be used includes determining a decrease in inflammatory cytokines and/or reduction in Treg cells in an animal model of autoimmune disease, such as a CIM mouse as disclosed herein in the Examples.

For example, a therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount is dependent of the desired therapeutic effect. For example, the therapeutically effective amount of an insl6 agent can be assessed in a mouse model of c protein-induced (poly)myositis (CIM) as disclosed herein and in the Examples, where a measure of the effective dose can be reduced muscle inflammation, e.g., reduced expression of any one or a combination of TNFα, MCP-1, CD11b, CD4 and/or CD8 in mice administered the Insl6 agent as compared to mice not administered the Insl6 agent.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. It is also noted that humans are treated generally longer than the mice or other experimental animals exemplified herein, which treatment has a length proportional to the length of the disease process and drug effectiveness. The doses may be single doses or multiple doses over a period of several days, but single doses are preferred.

In some embodiments, the Insl6 agents (e.g., proteins or nucleic acids encoding Insl6 protein and fragments thereof) can be administered to humans and other animals for therapy by any suitable route of administration, including orally, nasally, as by, for example, a spray, rectally, intravaginally, parenterally, intracisternally and topically, as by powders, ointments or drops, including buccally and sublingually.

After formulation with an appropriate pharmaceutically acceptable carrier in a desired dosage, a pharmaceutical composition comprising an Insl6 agent, e.g., Insl6 protein or functional fragment or variant thereof as disclosed herein can be administered to a subject. A pharmaceutical a composition comprising an Insl6 agent, e.g., Insl6 protein or functional fragment or variant thereof can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular, or nasal routes.

In a specific embodiment, it may be desirable to administer the pharmaceutical composition comprising an Insl6 polypeptide locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes. In some embodiments, Insl6 agents as disclosed herein are applied to the muscle using topical creams, patches, intramuscular injections and the like.

In some embodiments, an insl6 agent can be administered to a subject orally (e.g., in capsules, suspensions or tablets) or by parenteral administration. Conventional methods for oral administration include administering an Insl6 agent as a tablets, suspensions, solutions, emulsions, capsules, powders, syrups and the like are usable. Known techniques that deliver an insl6 agent orally or intravenously and retain the biological activity are preferred. Parenteral administration can include, for example, intramuscular, intravenous, intraarticular, intraarterial, intrathecal, subcutaneous, or intraperitoneal administration. The insl6 agent can also be administered orally, transdermally, topically, by inhalation (e.g., intrabronchial, intranasal, oral inhalation or intranasal drops) or rectally. Administration can be local or systemic as indicated. Agents, e.g., nucleic acid agents which encode an insl6 protein or variant or functional fragment thereof can also be delivered using a vector, e.g., a viral vector by methods which are well known to those skilled in the art.

When administering a composition comprising an Insl6 agent, e.g., Insl6 protein or functional fragment or variant thereof as disclosed herein parenterally, it will generally be formulated in a unit dosage injectable form (e.g., solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol), suitable mixtures thereof, and vegetable oils.

The term “Dosage unit” form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the Insl6 agent, e.g., Insl6 protein versus a nucleic acid (e.g., DNA, mRNA, MOD-RNA) encoding an Insl6 protein or functional fragment or variant thereof as disclosed herein and the particular therapeutic or prophylactic effect to be achieved, and (b) the limitations inherent in the art of compounding an Insl6 agent an active agent for the treatment of sensitivity in individuals.

The pharmaceutically acceptable compositions comprising an Insl6 agent, e.g., Insl6 protein or functional fragment or variant thereof as disclosed herein can be suspended in aqueous vehicles and introduced through conventional hypodermic needles or using infusion pumps.

### Pharmaceutical Compositions

In some embodiments, a composition comprising an Insl6 agent, e.g., Insl6 protein or functional fragment or variant thereof as disclosed herein can be formulated in any suitable means, e.g., as a sterile injectable solution, e.g., which can be prepared by incorporating the Insl6 agent in the required amount of the appropriate solvent with various of the other ingredients, as desired.

A pharmacological formulation of a composition comprising an Insl6 agent, e.g., Insl6 protein or functional fragment or variant thereof as disclosed herein can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include those presented in U.S. Pat. Nos. 5,225,182; 5,169,383; 5,167,616; 4,959,217; 4,925,678; 4,487,603; 4,486,194; 4,447,233; 4,447,224; 4,439,196 and 4,475,196. Other such implants, delivery systems, and modules are well known to those skilled in the art.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, e.g., parabens, chlorobutanol, phenol and sorbic acid. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

In another embodiment, a composition comprising an Insl6 agent, e.g., Insl6 protein or functional fragment or variant thereof as disclosed herein can comprise lipid-based formulations. Any of the known lipid-based drug delivery systems can be used in the practice of the invention. For instance, multivesicular liposomes, multilamellar liposomes and unilamellar liposomes can all be used so long as a sustained release rate of the encapsulated active compound can be established. Methods of making controlled release multivesicular liposome drug delivery systems are described in PCT Application Publication Nos: WO 9703652, WO 9513796, and WO 9423697, the contents of which are incorporated herein by reference.

The composition of the synthetic membrane vesicle is usually a combination of phospholipids, usually in combination with steroids, especially cholesterol. Other phospholipids or other lipids may also be used. Examples of lipids useful in synthetic membrane vesicle production include phosphatidylglycerols, phosphatidylcholines, phosphatidylserines, phosphatidylethanolamines, sphingolipids, cerebrosides, and gangliosides, with preferable embodiments including egg phosphatidylcholine, dipalmitoylphosphatidylcholine, distearoylphosphatidyleholine, dioleoylphosphatidylcholine, dipalmitoylphosphatidylglycerol, and dioleoylphosphatidylglycerol.

In preparing lipid-based vesicles containing an Insl6 agent, e.g., Insl6 protein or functional fragment or variant thereof, such variables as the efficiency of active compound encapsulation, liability of the active compound, homogeneity and size of the resulting population of vesicles, active compound-to-lipid ratio, permeability, instability of the preparation, and pharmaceutical acceptability of the formulation should be considered.

In another embodiment, the Insl6 polypeptide can be delivered in a vesicle, in particular a liposome (see Langer (1990) Science 249:1527-1533). In yet another embodiment, the Insl6 polypeptide can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding an Insl6 protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Prior to introduction, a composition comprising an Insl6 agent, e.g., Insl6 protein or functional fragment or variant thereof as disclosed herein can be sterilized, by any of the numerous available techniques of the art, such as with gamma radiation or electron beam sterilization.

In another embodiment of the invention, a composition comprising an Insl6 agent, e.g., Insl6 protein or functional fragment or variant thereof as disclosed herein, can be administered and/or formulated in conjunction (e.g., in combination) with any other therapeutic agent. For purpose of administration, an Insl6 agent, e.g., Insl6 protein or functional fragment or variant thereof as disclosed herein is preferably formulated as a pharmaceutical composition. Pharmaceutical compositions of the present invention comprise a compound of this invention and a pharmaceutically acceptable carrier, wherein the compound is present in the composition in an amount which is effective to treat the condition of interest. Appropriate concentrations and dosages can be readily determined by one skilled in the art.

Pharmaceutically acceptable carriers are familiar to those skilled in the art. For compositions formulated as liquid solutions, acceptable carriers include saline and sterile water, and may optionally include antioxidants, buffers, bacteriostats and other common additives. The compositions can also be formulated as pills, capsules, granules, or tablets which contain, in addition to a compound of this invention, diluents, dispersing and surface active agents, binders, and lubricants. One skilled in this art may further formulate the compounds of this invention in an appropriate manner, and in accordance with accepted practices, such as those disclosed in Remington's Pharmaceutical Sciences, Gennaro, Ed., Mack Publishing Co., Easton, Pa. 1990.

The compositions of the present invention can be in any form. These forms include, but are not limited to, solutions, suspensions, dispersions, ointments (including oral ointments), creams, pastes, gels, powders (including tooth powders), toothpastes, lozenges, salve, chewing gum, mouth sprays, pastilles, sachets, mouthwashes, aerosols, tablets, capsules, transdermal patches, that comprise one or more resolvins and/or protectins or their analogues of the invention.

Formulations of a composition comprising an Insl6 agent, e.g., Insl6 protein or functional fragment or variant thereof as disclosed herein can be prepared by a number or means known to persons skilled in the art. In some embodiments the formulations can be prepared for administration as an aerosol formulation, e.g., by combining (i) an Insl6 agent, e.g., Insl6 protein or functional fragment or variant thereof as disclosed herein in an amount sufficient to provide a plurality of therapeutically effective doses; (ii) the water addition in an amount effective to stabilize each of the formulations; (iii) the propellant in an amount sufficient to propel a plurality of doses from an aerosol canister; and (iv) any further optional components e.g. ethanol as a cosolvent; and dispersing the components. The components can be dispersed using a conventional mixer or homogenizer, by shaking, or by ultrasonic energy. Bulk formulation can be transferred to smaller individual aerosol vials by using valve to valve transfer methods, pressure filling or by using conventional cold-fill methods. It is not required that a stabilizer used in a suspension aerosol formulation be soluble in the propellant. Those that are not sufficiently soluble can be coated onto the drug particles in an appropriate amount and the coated particles can then be incorporated in a formulation as described above.

In certain embodiments, a composition comprising an Insl6 agent, which is a nucleic acid agent or polypeptide agent can be administered to a subject as a pharmaceutical composition with a pharmaceutically acceptable carrier. In certain embodiments, these pharmaceutical compositions optionally further comprise one or more additional therapeutic agents. In certain embodiments, the additional therapeutic agent or agents are autoimmune disease or drugs, such as immune suppressants and the like. In some embodiments, an additional therapeutic agent is a cortiosteriod. In some embodiments, an additional therapeutic agent is selected from the group consisting of Prednisone, methylprednisolone, Kenalog, Medrol Oral, Medrol (Pak) Oral, Depo-Medrol Inj, prednisolone Oral, Solu-Medrol Inj, hydrocortisone Oral, Cortef Oral, Solu-Medrol IV, cortisone Oral, Celestone Soluspan Inj, Orapred ODT Oral, Orapred Oral, Prelone Oral, methylprednisolone acetate Inj, Prednisone Intensol Oral, betamethasone acet & sod phos Inj, Veripred, Celestone Oral, methylprednisolone sodium succ IV, methylprednisolone sodium succ Inj, Millipred Oral, Solu-Medrol (PF) Inj, Solu-Cortef Inj, Aristospan Intra-Articular Inj, hydrocortisone sod succinate Inj, prednisolone sodium phosphate Oral, methylprednisolone sod suc(PF) IV, Solu-Medrol (PF) IV, triamcinolone hexacetonide Inj, A-Hydrocort Inj, A-Methapred Inj, Millipred DP Oral, Flo-Pred Oral, Aristospan Intralesional Inj, betamethasone Oral, methylprednisolone sod suc(PF) Inj, hydrocortisone sod succ (PF) Inj, Solu-Cortef (PF) Inj, prednisolone acetate Oral, dexamethasone in 0.9% NaCl IV, Rayos, levothyroxine. Of course, such therapeutic agents are which are known to those of ordinary skill in the art can readily be substituted as this list should not be considered exhaustive or limiting.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: water soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intravenous, oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs.

In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some instances, a composition comprising an Insl6 agent, e.g., Insl6 protein or functional fragment or variant thereof as disclosed herein can be in a formulation suitable for rectal or vaginal administration, for example as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore release the active compound. Suitable carriers and formulations for such administration are known in the art.

Dosage forms for the topical or transdermal administration of an Insl6 agent of this invention, e.g., for muscular administration include powders, sprays, ointments, pastes, creams, lotions, gels, solutions, patches and inhalants. An Insl6 agent, e.g., Insl6 protein or functional fragment or variant thereof as disclosed herein may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the Insl6 agents of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, an Insl6 agent which is Insl6 protein or functional fragment or variant thereof can be isolated and/or purified or substantially purified by one or more purification methods described herein or known by those skilled in the art. Generally, the purities are at least 90%, in particular 95% and often greater than 99%. In certain embodiments, the naturally occurring compound is excluded from the general description of the broader genus.

In some embodiments, the composition comprises at least one insl6 agent in combination with a pharmaceutically acceptable carrier. Some examples of materials which can serve as pharmaceutically acceptable carriers include, without limitation: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

In certain embodiments, a composition comprising an Insl6 agent, e.g., Insl6 protein or functional fragment or variant thereof as disclosed herein can contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention.

These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M., et al., “Pharmaceutical Salts,” J. Pharm. Sci., 1977; 66:1-19 which is incorporated herein by reference).

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of patients without undue toxicity, irritation, allergic response, and the like, commensurate with a reasonable benefit/risk ratio, and effective for their intended use. These compounds include the zwitterionic forms, where possible, of r compounds of the invention.

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “prodrug” refers to compounds or agents that are rapidly transformed in vivo to yield the active Insl6 agent, e.g., a biologically active or functional active Insl6 protein or nucleic acid (e.g., mRNA, DNA, MOD-RNA) which encodes a functionally active Insl6 protein. In some embodiments, insl6 prodrugs can be activated by hydrolysis in blood, e.g., via cleavage of a precursor Insl6 protein into an active Insl6 protein, similar to how insulin is activated from its proprotein into an active insulin protein. A thorough discussion is provided in T. Higachi and V. Stella, “Pro-drugs as Novel Delivery Systems,” Vol. 14 of the A.C.S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. The prodrug may be designed to alter the metabolic stability or the transport characteristics of an insl6 agent, to mask side effects or toxicity, or to alter other characteristics or properties of the Insl6 agent. By virtue of knowledge of pharmacodynamic processes and drug metabolism or post-translational protein processing of insl6 in vivo, once a pharmaceutically active compound is identified, those of skill in the pharmaceutical art generally can design insl6 agent prodrugs which can be activated in vivo to increase levels of Insl6 protein in the subject (see, e.g., Nogrady (1985) Medicinal Chemistry A Biochemical Approach, Oxford University Press, N.Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

As discussed herein, in some embodiments a composition comprising an Insl6 agent, e.g., Insl6 protein or functional fragment or variant thereof as disclosed herein can be conjugated or covalently attached to a targeting agent to increase their tissue specificity and targeting to a cell, for example a muscle cells. Targeting agents can include, for example without limitation, antibodies, cytokines and receptor ligands, as discussed in the section entitled “targeting.” In some embodiments, the targeting agent is overexpressed on the cells to be targeted, for example the muscle cells as compared to non-muscle cells.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of ordinary skill in the art.

### Gene Therapy

In some embodiments, where an insl6 agent is a nucleic acid encoding an insl6 protein or a functional derivative or functional variant or functional fragment thereof, it can be suitably administered as a vector, e.g., a viral vector.

In some embodiments, an insl6 agent is a nucleic acid encoding an insl6 protein or a functional derivative or functional variant or functional fragment thereof can be effectively used in treatment by gene therapy. See, generally, for example, U.S. Pat. No. 5,399,346, which is incorporated herein by reference. The general principle is to introduce the polynucleotide into a target cell in a patient, and where it is transcribed into the protein.

Entry into the cell can be facilitated by suitable techniques known in the art such as providing the polynucleotide in the form of a suitable vector, or encapsulation of the polynucleotide in a liposome.

A desired mode of gene therapy is to provide the polynucleotide in such a way that it will replicate inside the cell, enhancing and prolonging the desired effect. Thus, the polynucleotide is operably linked to a suitable promoter, such as the natural promoter of the corresponding gene, a heterologous promoter that is intrinsically active in liver, neuronal, bone, muscle, skin, joint, or cartilage cells, or a heterologous promoter that can be induced by a suitable agent.

Expression vectors compatible with eukaryotic cells, preferably those compatible with vertebrate cells, can be used to produce recombinant constructs for the expression of an Insl6 protein or a functional derivative or functional variant or functional fragment thereof as disclosed herein. Eukaryotic cell expression vectors are well known in the art and are available from several commercial sources. Typically, such vectors are provided containing convenient restriction sites for insertion of the desired DNA segment. These vectors can be viral vectors such as adenovirus, adeno-associated virus, pox virus such as an orthopox (vaccinia and attenuated vaccinia), avipox, lentivirus, murine moloney leukemia virus, etc. Alternatively, plasmid expression vectors can be used.

Viral vector systems which can be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. In a preferred embodiment, the vector is an adenovirus. Replication-defective viruses can also be advantageous.

The vector may or may not be incorporated into the cells genome. The constructs may include viral sequences for transfection, if desired. Alternatively, the construct may be incorporated into vectors capable of episomal replication, e.g., EPV and EBV vectors.

Constructs for the recombinant expression of an insl6 nucleic acid agent, e.g., DNA, MOD-RNA or RNAa, encoding an insl6 protein or a functional derivative or functional variant or functional fragment thereof as disclosed herein will generally be operatively linked to regulatory elements, e.g., promoters, enhancers, etc., to ensure the expression of the construct in target cells. Other specifics for vectors and constructs are described in further detail below.

Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Included in the term “regulatory elements” are nucleic acid sequences such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc. Regulatory sequences useful in the methods as disclosed herein are promoter elements which are sufficient to render promoter-dependent gene expression controllable for cell type-specific, tissue-specific or inducible by external signals or agents (e.g. enhancers or repressors); such elements may be located in the 5′ or 3′ regions of the native gene, or within an intron.

As used herein, the term “tissue-specific promoter” means a nucleic acid sequence that serves as a promoter, i.e., regulates expression of a selected nucleic acid sequence operably linked to the promoter, and which selectively affects expression of the selected nucleic acid sequence in specific cells of a tissue, such as cells of neural origin, e.g. neuronal cells.

In some embodiments, it can be advantageous to direct expression of an insl6 nucleic acid agent, e.g., DNA, MOD-RNA or RNAa, encoding an insl6 protein or a functional derivative or functional variant or functional fragment thereof as disclosed herein in a tissue- or cell-specific manner. Muscle-specific expression can be achieved, for example, using the skeletal muscle MKC promoter (as disclosed in U.S. Patent Application WO2007/100722, which is incorporated herein by reference), or other muscle-specific promoters, such as α-myosin heavy chain, myosin light chain-2 (which is specific for skeletal muscle (Shani et al., Nature, 314; 283-86, 1985), gonadotrophic releasing hormone gene control region which is active in the hypothalamus (Mason et al, Science, 234; 1372-78, 1986), and smooth muscle promoter SM22a, which are all commonly known in the art.

The term “constitutively active promoter” refers to a promoter of a gene which is expressed at all times within a given cell. Exemplary promoters for use in mammalian cells include cytomegalovirus (CMV), and for use in prokaryotic cells include the bacteriophage T7 and T3 promoters, and the like. The term “inducible promoter” refers to a promoter of a gene which can be expressed in response to a given signal, for example addition or reduction of an agent. Non-limiting examples of an inducible promoter are “tet-on” and “tet-off” promoters, or promoters that are regulated in a specific tissue type.

In a specific embodiment, viral vectors that contain nucleic acid sequences encoding an insl6 nucleic acid agent, e.g., DNA, MOD-RNA or RNAa, encoding an insl6 protein or a functional derivative or functional variant or functional fragment thereof as disclosed herein (including fusion proteins with fragments or derivatives or variants of Insl6 thereof as described herein) are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding a human Insl6 fusion polypeptide are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

The production of a recombinant retroviral vector carrying a gene of interest is typically achieved in two stages. First, sequence encoding an insl6 protein or a functional derivative or functional variant or functional fragment thereof, alone or fused to −Fc can be inserted into a retroviral vector which contains the sequences necessary for the efficient expression of the metabolic regulators (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (−PBS), a 3′ regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant retroviral vector, the vector DNA is introduced into a packaging cell line. Packaging cell lines provide viral proteins required in trans for the packaging of viral genomic RNA into viral particles having the desired host range (e.g., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permits entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles which contain viral genomic RNA derived from one virus encapsidated by the envelope proteins of another virus are said to be pseudotyped virus particles.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia virus, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In another embodiment, lentiviral vectors are used, such as the HIV based vectors described in U.S. Pat. Nos. 6,143,520; 5,665,557; and 5,981,276, which are herein incorporated by reference. Use of Adeno-associated virus (AAV) vectors is also contemplated (Walsh et al., Proc. Soc. Exp. Biol. Med. 204:289-300 (1993); U.S. Pat. No. 5,436,146 which is incorporated herein by reference).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

U.S. Pat. No. 5,676,954 (which is herein incorporated by reference) reports on the injection of genetic material, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein incorporated by reference) provide methods for delivering DNA-cationic lipid complexes to mammals. Such cationic lipid complexes or nanoparticles can also be used to deliver protein.

A gene or nucleic acid sequence can be introduced into a target cell by any suitable method. For example, an human Insl6 protein, or Insl6-Fc fusion polypeptide construct can be introduced into a cell by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like. A nucleic acid encoding an human Insl6 fusion polypeptide can be introduced into cells by electroporation (see, e.g., Wong and Neumann, Biochem. Biophys. Res. Commun. 107:584-87 (1982)) and biolistics (e.g., a gene gun; Johnston and Tang, Methods Cell Biol. 43 Pt A:353-65 (1994); Fynan et al., Proc. Natl. Acad. Sci. USA 90:11478-82 (1993)).

In certain embodiments, a gene or nucleic acid sequence encoding human Insl6 protein, or Insl6-Fc fusion polypeptide can be introduced into target cells by transfection or lipofection. Suitable agents for transfection or lipofection include, for example, calcium phosphate, DEAE dextran, lipofectin, lipfectamine, DIMRIE C, Superfect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

Methods known in the art for the therapeutic delivery of agents such as proteins and/or nucleic acids can be used for the delivery of a polypeptide or nucleic acid encoding an human Insl6 protein, or Insl6-Fc fusion polypeptide for modulating iron metabolism and/or for increasing serum iron concentration in a subject, e.g., cellular transfection, gene therapy, direct administration with a delivery vehicle or pharmaceutically acceptable carrier, indirect delivery by providing recombinant cells comprising a nucleic acid encoding a targeting fusion polypeptide of the invention.

Various delivery systems are known and can be used to directly administer therapeutic polypeptides such as the human Insl6 protein, or Insl6-Fc fusion polypeptide and/or a nucleic acid encoding a human Insl6 protein, or Insl6-Fc fusion fusion polypeptide as disclosed herein, e.g., encapsulation in liposomes, microparticles, microcapsules, recombinant cells capable of expressing the compound, and receptor-mediated endocytosis (see, e.g., Wu and Wu, 1987, J. Biol. Chem. 262:4429-4432). Methods of introduction can be enteral or parenteral and include but are not limited to intradermal, intramuscular, intraperitoneal, intravenous, subcutaneous, pulmonary, intranasal, intraocular, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105).

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked human Insl6 protein, or Insl6-Fc fusion polypeptide encoding nucleic acid segment into the selected expression/delivery vector, many equivalent vectors for the practice of the methods described herein can be generated.

It will be appreciated by those of skill that cloned genes readily can be manipulated to alter the amino acid sequence of a protein. The cloned gene for human Insl6 protein, or Insl6-Fc fusion that comprise part of the Insl6 fusion polypeptide can be manipulated by a variety of well known techniques for in vitro mutagenesis, among others, to produce variants of the naturally occurring human protein, herein referred to as muteins or variants or mutants of Insl6, which may be used in accordance with the methods and compositions described herein.

The variation in primary structure of muteins of Insl6 useful in the invention, for instance, may include deletions, additions and substitutions. The substitutions may be conservative or non-conservative. The differences between the natural protein and the mutein generally conserve desired properties, mitigate or eliminate undesired properties and add desired or new properties.

Remington's Pharmaceutical sciences Ed. Germany, Merk Publishing, Easton, Pa., 1995 (the contents of which are hereby incorporated by reference), discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; malt; gelatin; talc; excipients such as cocoa butter and: suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; water; isotonic saline; Ringer's solution, ethyl alcohol, and phosphate buffer solutions, as well as other non-toxic compatible lubricants such as sodium lauryl sulfate and magnesium sulfate, as well as coloring agents, releasing agents, coating agents, sweetening, flavoring and perfuming agents, preservatives and antioxidants can also be present in the composition, according to the judgment of the formulator.

### Kits

The invention also provides kits or pharmaceutical packages that comprise an Insl6 agent, e.g., an Insl6 protein, or functional variant or functional fragment or fusion protein thereof for use in the prevention and/or treatment of an autoimmune diseases or immune related disorder as described herein. The kit can comprise the Insl6 agent composition in the form of, for example, tablets, capsules, or lyophilized powders, and can optionally include instructions for using the Insl6 agent for the treatment of an autoimmune diseases or immune related disorder. A composition of an Insl6 agent, e.g., an Insl6 protein, or functional variant or functional fragment or fusion protein thereof can be provided in the kits or packages in a bottle or another appropriate form (e.g., a blister pack). Optionally, the kits or pharmaceutical packages can also include other pharmaceutically active agents (see, e.g., the agents listed above, such as other agents used for treatment of autoimmune diseases and disorders), and/or materials used in administration of the drug(s), such as diluents, needles, syringes, applicators, and the like.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

Some embodiments of the present invention can be defined as any of the following numbered paragraphs:

1. A method for treating an autoimmune disease in a subject comprising administering to the subject a composition comprising an insulin-like 6 (Insl6) protein or fragment thereof.
2. A method for treating an autoimmune disease in a subject comprising administering to the subject determined to have an autoimmune disease a composition comprising a insulin-like 6 (Insl6) protein or fragment thereof, and wherein the composition is not administered to a subject where the subject is not determined to have an autoimmune disease.
3. The method of paragraph 1 or 2, wherein the autoimmune disease is myositis.
4. The method of paragraph 1 or 2, wherein the autoimmune disease is a T-cell mediated autoimmune disease.
5. The method of paragraph 1, wherein the myositis is selected from any of the diseases consisting of: polymyositis (PM), dermatomyositis (DM) or inclusion body myositis (IBM).
6. The method of paragraph 1 or 2, wherein the autoimmune disease is selected from the group consisting of: Addison's disease, Celiac disease—sprue (gluten-sensitive enteropathy), Dermatomyositis, Graves disease, Hashimoto's thyroiditis, Multiple sclerosis, Myasthenia gravis, Pernicious anemia; Reactive arthritis, Rheumatoid arthritis, Sjogren syndrome, Systemic lupus, erythematosus, and Type I diabetes.
7. The method of any of paragraphs 1 to 6, wherein the subject has elevated T-regulatory (T_{reg}) cells as compared to a subject without an autoimmune disease.
8. The method of paragraph 7, wherein the T-regulatory (T_{reg}) cells express CD4, CD25 and Foxp3.
9. The method of paragraph 8, wherein the T-regulatory (T_{reg}) cells further express CTLA-4 and αβTCR.
10. The method of any of paragraphs 1 to 9, wherein the subject has a decreased level of Insl6 protein by a statistically significant amount in a biological sample obtained from the subject as compared to a reference level of Insl6 protein.
11. The method of any of paragraphs 1 to 10, wherein the subject has a decreased level of Insl6 mRNA by a statistically significant amount in a biological sample obtained from the subject as compared to a reference level of Insl6 mRNA.
12. The method of paragraph 11, wherein the level of Insl6 mRNA is measured using RT-PCR.
13. The method of any of paragraphs 10 to 12, wherein the biological sample is a muscle biopsy sample.
14. The method of paragraph 1 or 2, wherein the fragment of insulin-like 6 protein is a biologically active fragment of Insl6.
15. The method of paragraph 14, wherein a biologically active fragment of Insl6 comprises at least the B-domain corresponding to amino acids 21-53 of SEQ ID NO:1 or a functional fragment thereof.
16. The method of paragraph 14, wherein a biologically active fragment of Insl6 comprises at least the A-domain corresponding to amino acids 173-198 of SEQ ID NO:1 or a functional fragment thereof.
17. The method of paragraph 14, wherein a biologically active fragment of Insl6 comprises the B-domain corresponding to amino acids 21-53 of SEQ ID NO:1 and A-domain corresponding to amino acids 173-198 of SEQ ID NO:1 or a functional fragments thereof.
18. The method of any of paragraphs 1 to 17, wherein the insulin-like 6 (Insl6) protein or Insl6 fragment is fused to a carrier protein.
19. The method of any of paragraphs 1 to 18, wherein the insulin-like 6 (Insl6) protein or Insl6 fragment is fused to a Fc.
20. The method of any of paragraphs 1 to 19, wherein the insulin-like 6 (insl6) protein is encoded by nucleic acid present in a vector.
21. The method of any of paragraphs 1 to 20, wherein the vector is a viral vector.
22. The method of any of paragraphs 1 to 21, wherein the composition further comprises an additional therapeutic agent to treat myositis.
23. The method of any of paragraphs 1 to 22, wherein the additional therapeutic agent is a corticosteroid.
24. The method of any of paragraphs 1 to 23, wherein the insulin-like 6 (Insl6) protein or biologically active fragment thereof reduces at least one inflammatory protein selected from the group consisting of: CD4, CD8, CD11b, TNFα and MCP-1.
25. The method of any of paragraphs 1 to 24, wherein administration is by topical administration, parenteral administration, enteral oral, parenteral (e.g., intravenous, subcutaneous or intramuscular), rectal, intracisternal, intravaginal, intraperitoneal, ocular, or nasal routes.
26. A fusion protein comprising:
a. an insulin-like 6 (Insl6) polypeptide or fragment thereof, wherein said fragment has at least 95% amino acid sequence identity to a portion of the Insl6 protein and is at least 6 amino acids; and
b. a first fusion partner, wherein said first fusion partner is conjugated to said Insl6 polypeptide or fragment thereof.
27. The fusion protein of paragraph 26, wherein said first fusion partner is fused to the N-terminus or to the C-terminus of the Insl6 protein or Insl6 fragment.
28. The fusion protein of paragraph 26, wherein said first fusion partner is IgG1 Fc.
29. The fusion protein of paragraph 28, wherein said IgG1 Fc is human IgG1 Fc.
30. The fusion protein of paragraph 26, wherein said Insl6 fragment is a soluble fragment.
31. The fusion protein of paragraph 26, further comprising a second fusion partner.
32. The fusion protein of paragraph 26, wherein the conjugation is a covalent bond.
33. The fusion protein of paragraph 26, wherein said Insl6 fragment lacks the N-terminal signal sequence corresponding to amino acids 1-20 of SEQ ID NO:1.
34. The fusion protein of paragraph 26, wherein said Insl6 protein is a human Insl6 protein.
35. The fusion protein of paragraph 34, wherein the human Insl6 protein corresponds to amino acid SEQ ID NO: 1, or a functional variant or functional derivative or functional fragment thereof.
36. The fusion protein of paragraph 35, wherein a functional fragment of Insl6 comprises at least the B-domain corresponding to amino acids 21-53 of SEQ ID NO:1 or a functional fragment thereof.
37. The fusion protein of paragraph 35, wherein a functional fragment of Insl6 comprises at least the A-domain corresponding to amino acids 173-198 of SEQ ID NO:1 or a functional fragment thereof.
38. The fusion protein of paragraph 35, wherein a functional fragment of Insl6 comprises the B-domain corresponding to amino acids 21-53 of SEQ ID NO:1 and A-domain corresponding to amino acids 173-198 of SEQ ID NO:1 or a functional fragments thereof.
39. The fusion protein of paragraph 26, wherein said fusion protein has enhanced proteolytic stability.
40. The fusion protein of any of paragraphs 26 to 39, wherein said fusion protein has an amino acid sequence with at least 95% identity to the sequence of SEQ ID NO: 1, or a functional derivative or functional variant thereof.
41. The fusion protein of any of paragraphs 36 to 38, wherein said fusion protein has an amino acid sequence with at least 95% identity to amino acids 21-53 or amino acids 173-198 of SEQ ID NO: 1.
42. The fusion protein of paragraph 35, wherein said fusion protein has an amino acid sequence comprising the sequence of SEQ ID NO: 1, or a functional derivative or functional variant thereof.
43. The fusion protein of paragraph 35, wherein said fusion protein has an amino acid sequence consisting essentially of the sequence of SEQ ID NO: 1.
44. The fusion protein of any of the paragraphs 26 to 43 for treating an inflammatory disorder or an autoimmune disease.
45. The fusion protein of claim 44, wherein the autoimmune disease is myositis.
46. The fusion protein of claim 45, wherein the myositis is selected from any of the diseases consisting of: polymyositis (PM), dermatomyositis (DM) or inclusion body myositis (IBM).
47. A pharmaceutical composition comprising the fusion protein of any of claims 26 to 46 and a pharmaceutically acceptable carrier.
48. A method for producing the fusion protein of any of the claims 26 to 45 comprising:
a. introducing into a cell with a vector comprising a sequence encoding the fusion protein operably linked to a promoter, wherein the fusion protein is insulin-like 6 (Insl6) polypeptide or fragment thereof linked to a first fusion protein; and
b. culturing said cell under conditions where said protein is expressed.
49. The method of paragraph 48, further comprising purifying said protein of step (b).
50. A polynucleotide encoding the fusion protein of any of paragraphs 26 to 46, wherein the polynucleotide encodes an insulin-like 6 (Insl6) polypeptide or fragment thereof which has at least 95% amino acid sequence identity to a portion of the insulin-like 6 (Insl6) polypeptide or fragment thereof protein; and a first fusion partner.
51. A polynucleotide of paragraph 50, wherein the polynucleotide encodes at least the B-domain of Insl6, which has at least 95% amino acid sequence identity to amino acids 21-53 of SEQ ID NO: 1.
52. A polynucleotide of paragraph 50, wherein the polynucleotide encodes at least the A-domain of Insl6, which has at least 95% amino acid sequence identity to amino acids 173-198 of SEQ ID NO: 1.
53. A vector comprising the polynucleotide of paragraph 50.
54. The vector of paragraph 53, wherein the vector is a viral vector.
55. The vector of paragraph 54, wherein the viral vector is selected from the group consisting of an adenoviral vector, a poxvirus vector and a lentiviral vector.
56. The vector of any of paragraphs 53 to 55, wherein the nucleic acid sequence encodes an insulin-like 6 (Insl6) polypeptide or fragment thereof which has at least 95% amino acid sequence identity to a portion of the Insl6 protein; and a first fusion partner, wherein the nucleic acid sequence is operatively linked to tissue- or cell-type specific promoter.
57. A vector of paragraph 56, wherein the nucleic acid sequence encodes at least the B-domain of Insl6, which has at least 95% amino acid sequence identity to amino acids 21-53 of SEQ ID NO: 1, and a first fusion partner, wherein the nucleic acid sequence is operatively linked to tissue- or cell-type specific promoter.
58. A vector of paragraph 56, wherein the nucleic acid sequence encodes at least the A-domain of Insl6, which has at least 95% amino acid sequence identity to amino acids 173-198 of SEQ ID NO: 1, and a first fusion partner, wherein the nucleic acid sequence is operatively linked to tissue- or cell-type specific promoter.
59. The vector of any of paragraphs 56 to 58, wherein the tissue- or cell-type specific promoter is a muscle specific promoter.
60. A pharmaceutical composition comprising the vector of any of the paragraphs 53 to 59 and a pharmaceutically acceptable carrier.
61. A pharmaceutical composition comprising the vector of any of the paragraphs 53 to 59.
62. A host cell comprising the vector of any of the paragraphs 53 to 59.
63. A method for treating a subject having a muscle inflammatory disorder or an autoimmune disease, said method comprising administering to said subject the fusion protein of any of paragraphs 26 to 46 in an amount effective to treat said subject.
64. The method of paragraph 63, wherein the muscle inflammatory disorder is myositis.
65. The method of paragraph 64, wherein the myositis is selected from any of the diseases consisting of: polymyositis (PM), dermatomyositis (DM) or inclusion body myositis (IBM).

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

The invention can be understood more fully by reference to the following detailed description and illustrative examples, that are intended to exemplify non-limiting embodiments of the invention.

### EXAMPLES

The following examples are provided for illustrative purposes only and are not intended to limit the scope of the invention.

The description of the present invention has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiment described and shown in the figures was chosen and described in order to best explain the principles of the invention, the practical application, and to enable others of ordinary skill in the art to understand the invention for various embodiments with various modifications as are suited to the particular use contemplated.

Materials and Methods:

Rotarod Testing:

For treadmill testing, the slope is 0 degrees (horizontal) and mice are acclimatized by running at 10 meters per minute for 5 minutes. To determine running distance, the treadmill speed is increased from the acclimatization speed by 2 meters per minute every five minutes, and the running distance until exhaustion is recorded. For the rotarod test, mice are acclimatized at 10 rpm for 5 minutes. Following acclimatization, the length of time on the rotarod is recorded when it is run at 20 rpm (a score of 1 is defined as 30 seconds on the rotarod for 30 seconds at 20 rpm). The results are presented as the mean±SEM.

### Example 1

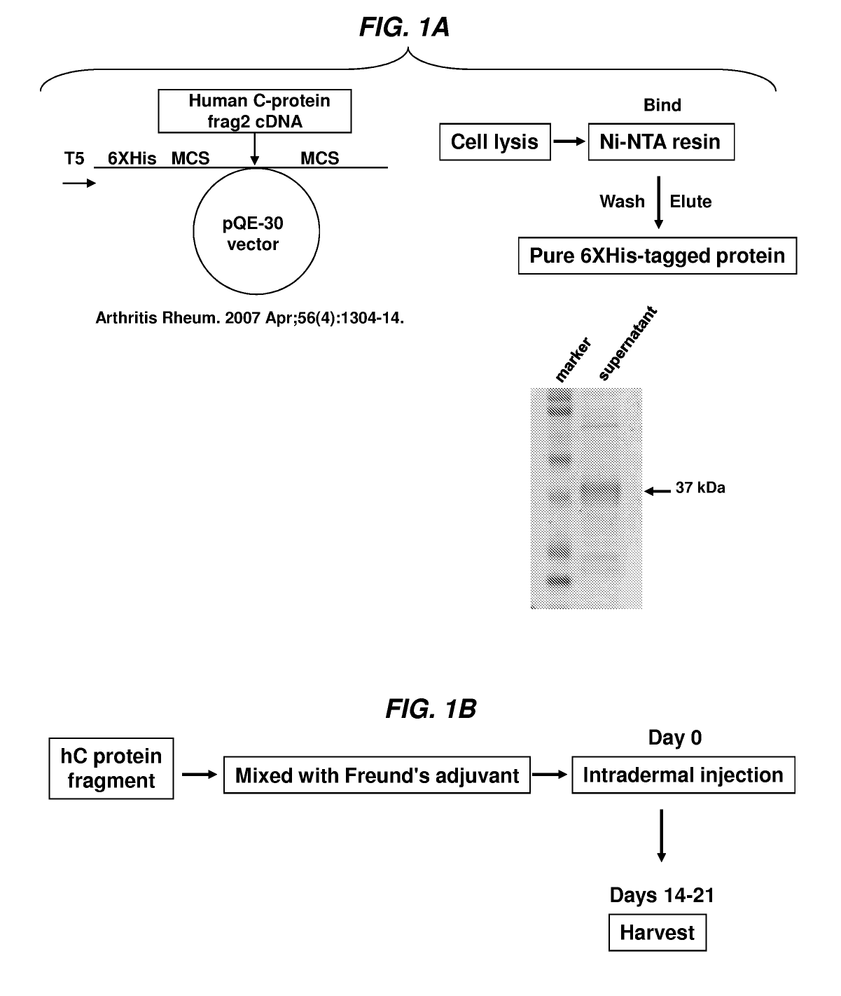

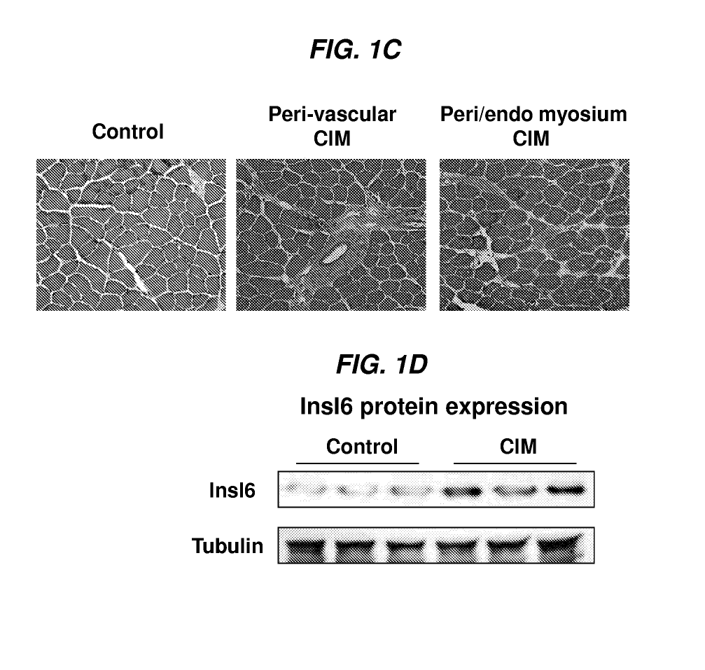

A model of C protein-induced (poly)myositis model (CIM) was established as described previously (T. Sugihara, et al. 2007 Arthritis Rheum 56:1304). This model leads to muscle tissue inflammation (FIG. 1) and muscle weakness in rotarod and treadmill evaluations (FIG. 2). Histological analysis of tissue indicates that mononuclear cell infiltration occurs in the perimysial (surrounding myofibers) and perivascular (surrounding vessels) sites, but rarely seen in endomysial sites (within myofibers) (FIG. 1).

Mice that overexpress the murine Insulin-like 6 (Insl6) transgene (TG), described previously (L. Zeng et al. 2010J Biol. Chem. 285:36060), were evaluated relative to wild-type mice in the CIM model. Insl6 overexpressing mice performed significantly better than wild-type mice in both rotarod and treadmill assessments of muscle performance (FIG. 2).

Inflammation is reduced in the muscle-specific Insl6-overexpressing mice in the CIM model (FIG. 3). Wild-type (WT) and Insl6 transgenic (TG) mice were immunized as described in the CIM protocol (FIG. 1). At 14 days post-immunization, mice were sacrificed and total RNA was isolated from tibialis anterior muscle to measure relative transcript expression by qRT-PCR. Transcripts encoding inflammatory proteins and markers, including CD4, CD8, CD11b, TNF-α and MCP-1, were significantly reduced in the mice that overexpress Insl6 in muscle (FIG. 3).

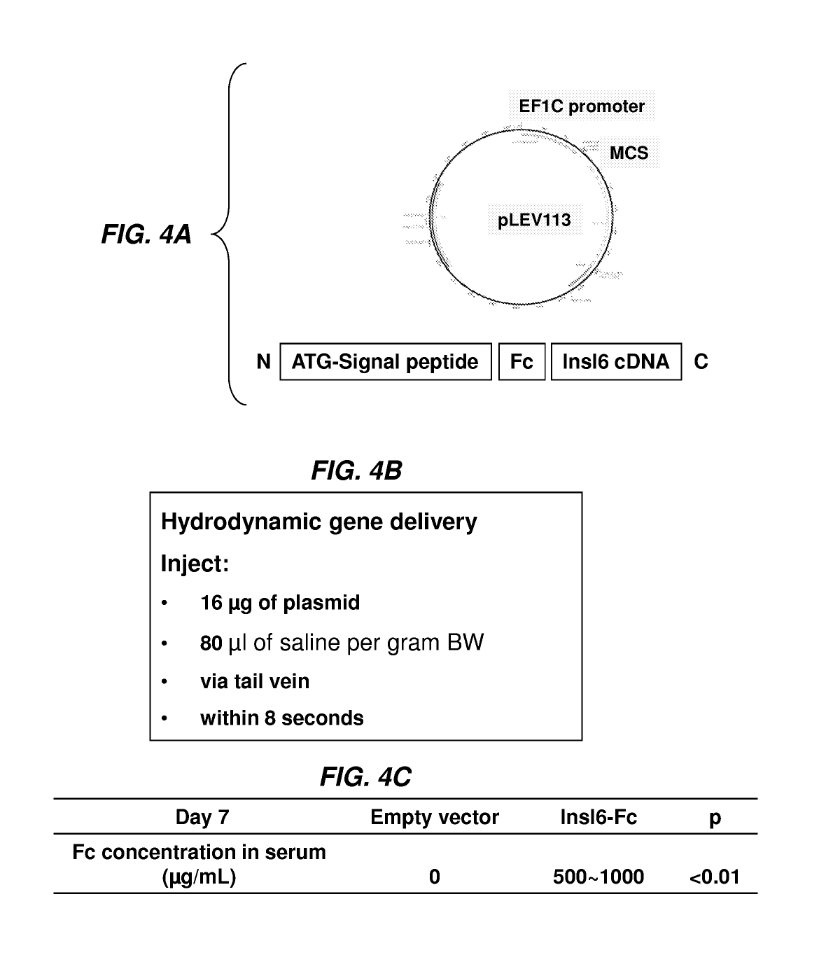

A plasmid expressing an Fc-Insl6 fusion protein was constructed for hydrodynamic tail vein delivery to mice (FIG. 4). This plasmid encodes the IgG Fc fragment fused to the N-terminus of murine Insl6. The Fc fragment is commonly fused with proteins to increase their serum half-life (e.g. I. Shiojima et al. 2005 J Clin Invest. 115:2108). The hydrodynamic delivery was performed according to the published procedure (M. G. Sebstyen et al. 2006 J. Gene Med 8:852). For each mouse, 16 micrograms of plasmid, in 80 microliters of volume per gram of body weight, was delivered within 8 seconds via tail vein injection. Control experiments were conducted with the empty plasmid vector (no transgene). ELISA analysis of the serum indicated circulating levels of Fc-containing protein at 7 days after hydrodynamic delivery in mice that had received the plasmid encoding the Fc-Insl6 encoding plasmid.

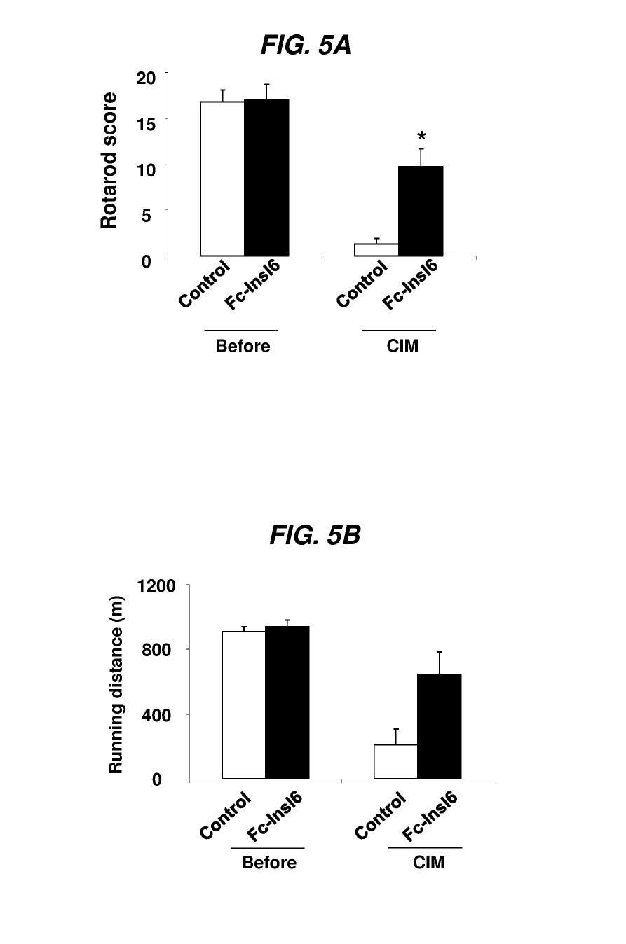

To corroborate findings with the Insl6 transgenic mouse, hydrodynamic delivery of the plasmid expressing the Fc-Insl6 fusion was performed after the mice were immunized to develop myositis. Mice receiving the Fc-Insl6 expressing plasmid performed significantly better in the rotarod and treadmill tests (FIG. 5). Insl6 transgenic (TG) mice were also demonstrated to have more Foxp3+ regulatory T cells in cardiotoxin-injured muscles as determined by immunohistochemistry. Cardiotoxin was injected into tibialis anterior (TA) muscle of Insl6 TG male mice and their wild-type littermates. Immunostaning with anti-Foxp3 of the TA muscle 7 days after cardiotoxin injection indicated more T_{reg} cells (data not shown).

Insl6 transcript levels were determined in a cohort of myositis patients and control subject that were matched for age and gender. Patients with polymyositis (PM) or dermatomyositis (DM) displayed lower level of the Insl6 transcript (FIG. 6).

### REFERENCES

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference. Accordingly, the references are each incorporated herein in their entirety by reference.

1. A method for treating an autoimmune disease in a subject comprising administering an effective amount of a composition comprising a insulin-like 6 (Insl6) protein or a biologically active fragment thereof to the subject, thereby reducing T-cell activation, B-cell activation or T-cell and B-cell activation.

2-65. (canceled)

66. The method of claim 1, wherein the composition is administered to a subject who has been identified as having an autoimmune disease, and wherein the composition is not administered to a subject where the subject is not determined to have an autoimmune disease.

67. The method of claim 1, wherein the autoimmune disease is myositis or a T-cell mediated autoimmune disease or a muscle inflammatory disorder.

68. The method of claim 67, wherein the myositis is selected from any of the diseases consisting of: polymyositis (PM), dermatomyositis (DM) or inclusion body myositis (IBM).

69. The method of claim 1, wherein the autoimmune disease is selected from the group consisting of: Addison's disease, Celiac disease—sprue (gluten-sensitive enteropathy), Dermatomyositis, Graves disease, Hashimoto's thyroiditis, Multiple sclerosis, Myasthenia gravis, Pernicious anemia; Reactive arthritis, Rheumatoid arthritis, Sjogren syndrome, Systemic lupus, erythematosus, and Type I diabetes.

70. The method of claim 1, wherein the subject has elevated T-regulatory (T_{reg}) cells as compared to a subject without an autoimmune disease.

71. The method of claim 1, wherein the subject has a decreased level of Insl6 protein or mRNA as determined by measuring the amount in a biological sample obtained from the subject and that to a reference amount.

72. The method of claim 1, wherein a biologically active fragment of Insl6 is selected from the group consisting of: at least the B-domain corresponding to amino acids 21-53 of SEQ ID NO:1, or at least the A-domain corresponding to amino acids 173-198 of SEQ ID NO:1, or at least the B-domain corresponding to amino acids 21-53 of SEQ ID NO:1 and at least the A-domain corresponding to amino acids 173-198 of SEQ ID NO:1.

73. The method of claim 1, wherein the insulin-like 6 (Insl6) protein or Insl6 fragment is fused to a carrier protein.

74. The method of claim 1, wherein the insulin-like 6 (Insl6) protein or Insl6 fragment is fused to a Fc.

75. The method of claim 73, wherein the insulin-like 6 (insl6) protein or Insl6 fragment fused to a carrier protein is encoded by nucleic acid present in a vector.

76. A fusion protein comprising:
a. an insulin-like 6 (Insl6) polypeptide or fragment thereof, wherein said fragment has at least 95% amino acid sequence identity to a portion of the Insl6 protein and is at least 6 amino acids; and
b. a first fusion partner, wherein said first fusion partner is conjugated to said Insl6 polypeptide or fragment thereof.

77. The fusion protein of claim 76, wherein said first fusion partner is fused to the N-terminus or to the C-terminus of the Insl6 protein or Insl6 fragment.

78. The fusion protein of claim 76, wherein said first fusion partner is IgG1 Fc.

79. The fusion protein of claim 76, wherein said Insl6 fragment is a soluble fragment.

80. The fusion protein of claim 76, further comprising a second fusion partner.

81. The fusion protein of claim 76, wherein said Insl6 fragment lacks the N-terminal signal sequence corresponding to amino acids 1-20 of SEQ ID NO:1.

82. The fusion protein of claim 76, wherein the Insl6 protein corresponds to amino acid SEQ ID NO: 1, or a functional variant or functional derivative or functional fragment thereof.

83. The fusion protein of claim 82, wherein a functional fragment of Insl6 is selected from the group consisting of: at least the B-domain corresponding to amino acids 21-53 of SEQ ID NO:1, or at least the A-domain corresponding to amino acids 173-198 of SEQ ID NO:1, or at least the B-domain corresponding to amino acids 21-53 of SEQ ID NO:1 and at least the A-domain corresponding to amino acids 173-198 of SEQ ID NO:1, or functional fragments thereof.

84. The fusion protein of claim 76, wherein said fusion protein has an amino acid sequence with at least 95% identity to the sequence of SEQ ID NO: 1, or an amino acid sequence with at least 95% identity to amino acids 21-53 or amino acids 173-198 of SEQ ID NO: 1, or a functional derivative or functional variant thereof.

85. The method of claim 1, comprising administering the fusion protein of claim 77.

86. A pharmaceutical composition comprising the fusion protein of claim 76 and a pharmaceutically acceptable carrier.

87. A polynucleotide encoding the fusion protein of claim 76, wherein the polynucleotide encodes an insulin-like 6 (Insl6) polypeptide or fragment thereof which has at least 95% amino acid sequence identity to a portion of the insulin-like 6 (Insl6) polypeptide or fragment thereof; and a first fusion partner.

88. A polynucleotide of claim 87, wherein the polynucleotide encodes a fragment of the Insl6 polypeptide comprising at least the B-domain of Insl6, which has at least 95% amino acid sequence identity to amino acids 21-53 of SEQ ID NO: 1, or encodes a fragment of the Insl6 polypeptide comprising at least the A-domain of Insl6, which has at least 95% amino acid sequence identity to amino acids 173-198 of SEQ ID NO: 1.

89. A vector comprising the polynucleotide of claim 87.

90. The vector of claim 89, wherein the polynucleotide is operatively linked to tissue- or cell-type specific promoter.

91. The vector of claim 90, wherein the tissue- or cell-type specific promoter is a muscle specific promoter.

92. A host cell comprising the vector of claim 89.


SEQUENCE LISTING (4 sequences)

SEQ ID NO: 1 - protein, 213 aa, Homo sapiens
MPRLLRLSLLWLGLLLVRFSRELSDISSARKLCGRYLVKEIEKLCGHANWSQFRFEEETPFSRLIAQASEKVEAYSPYQFESPQTASPARGRGTNPVSTSWEEAVNSWEMQSLPEYKDKKGYSPLGKTREFSSSHNINVYIHENAKFQKKRRNKIKTLSNLFWGHHPQRKRRGYSEKCCLTGCTKEELSIACLPYIDFKRLKEKRSSLVTKIY

SEQ ID NO: 2 - DNA, 711 bp, Homo sapiens
gcctggggtcacagggatgccgcggctcctccgcttgtccctgctgtggcttggactcctgctggttcggttttctcgtgaactgagcgacatcagcagtgccaggaagctgtgcggcaggtacttggtgaaagaaatagaaaaactctgcggccatgccaactggagccagttccgtttcgaggaggaaacccctttctcacggttgattgcacaggcctcggagaaggtcgaagcctacagcccataccagttcgaaagcccgcaaaccgcttccccggcccggggaagaggcacaaacccagtgtctacttcttgggaagaagcagtaaacagttgggaaatgcagtcactacctgagtataaggataaaaagggatattcaccccttggtaagacaagagaattttcttcatcacataatatcaatgtatatattcatgagaatgcaaaatttcagaagaaacgtagaaacaaaattaaaaccttaagcaatttgttttgggggcatcatccccaaagaaaacgcagaggatattcagaaaagtgttgtcttacaggatgtacaaaagaagaacttagcattgcatgtcttccatatattgattttaaaaggctaaaggaaaaaagatcatcacttgtaactaagatatactaaccatcttagaattttttctaacctaataaaagcttaatacatttatttaaaaa

SEQ ID NO: 3 - protein, 252 aa, Homo sapiens
LELVPRGSGDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 4 - protein, 6 aa, Artificial Sequence
  Description of Artificial Sequence Synthetic 6xHis tag
HHHHHH